(19) United States
(12) Patent Application Publication
(10) Pub. No.: US 2024/0385185 A1
(43) Pub. Date: Nov. 21, 2024

(54) METHODS AND SYSTEMS FOR DETECTING URINARY TRACT INFECTIONS

(71) Applicant: CAP Diagnostics, LLC dba Pathnostics, Irvine, CA (US)

(72) Inventors: David A. Baunoch, Irvine, CA (US); Natalie Luke, Irvine, CA (US); Richard Festa, Irvine, CA (US); Mohit Mathur, Irvine, CA (US)

(21) Appl. No.: 18/669,370

(22) Filed: May 20, 2024

Related U.S. Application Data
(60) Provisional application No. 63/503,393, filed on May 19, 2023.
(60) Provisional application No. 63/514,875, filed on Jul. 21, 2023.

(51) Int. Cl. G01N 33/543; G01N 33/68; G16H 50/20

(52) U.S. Cl. CPC G01N 33/54388; G01N 33/6869; G01N 33/6893; G16H 50/20; G01N 2333/5421; G01N 2800/348

(57) ABSTRACT

Current UTI diagnostics face sensitivity challenges, especially with fastidious organisms and polymicrobial infections. Diagnosing UTIs in geriatric and pediatric patients, including those with communication difficulties like dementia, adds complexity due to atypical symptoms. Thus, systems, devices, and methods for UTI diagnosis across diverse patient groups are described herein. These methods utilize urinary biomarkers (NGAL, IL-8, IL-1β) to differentiate UTIs from asymptomatic bacteriuria. A consensus criterion, requiring≥2 biomarkers, achieves high sensitivity (84.0%), specificity (91.2%), and accuracy (86.9%). This biomarker consensus enhances UTI diagnosis in compact diagnostic systems, especially when standard urine culture and multiplex PCR results diverge.

### CROSS-REFERENCE TO RELATED APPLICATIONS

This application is a non-provisional and claims benefit of U.S. Provisional Application No. 63/514,875 filed Jul. 21, 2023, and U.S. Provisional Application No. 63/503,393 filed May 19, 2023, the specifications of which are incorporated herein in their entirety by reference.

### FIELD OF THE INVENTION

The present invention features a point-of-care device designed to indicate a patient's immune response to pathogens in the urinary tract, such as those associated with urinary tract infections. The methods, systems, and devices herein focus on the detection of UTI-associated biomarkers, including neutrophil gelatinase-associated lipocalin (NGAL), interleukin-8 (IL-8), and interleukin-1-beta (IL-1β). These biomarkers, when detected above specific thresholds in urine, collectively serve as strong indicators of the patient's immune response, leading to a more accurate diagnosis of UTI and determining the necessity for follow-up testing.

### BACKGROUND OF THE INVENTION

The use of standard urine culture (SUC) to identify classical uropathogens in urinary tract infection (UTI) has been standard practice for several decades, but has several limitations. One such limitation is that SUC uses specific media and conditions that result in cultivating easy-to-grow microbes like Escherichia coli (E. coli) yet poorly grows non-E. coli pathogens which have been reported as important emerging uropathogens. Recent studies have increased awareness of many additional clinically relevant microbial species, such as gram-positive organisms, fastidious microbes, and fungi, which can contribute to urinary microbiome dysbiosis in symptomatic subjects. Additionally, studies using more sensitive culture techniques, such as enhanced-quantitative urine culture (EQUC), and culture-free methods such as gene sequencing and MALDI-TOF have also led to the discovery of the uromicrobiome, which is present even in asymptomatic individuals.

The limitations of SUC, the presence of a urinary microbiome, and the high prevalence of asymptomatic bacteriuria underscore the need to develop diagnostic tests that can identify the presence of urinary tract inflammation in UTI symptomatic patients with high sensitivity and specificity. First, these tests will help identify patients with false negative SUC results who are still likely to have a UTI and need appropriate therapy. Second, while the identification of uropathogens with more sensitive tests such as multiplex polymerase chain reaction (M-PCR) is a strong indicator of infection, there remain questions about whether microbes detected using these tests are associated with a UTI and cause inflammation of the urinary tract. Accurate tests that identify true UTI patients would also be important in pediatric cases where symptom elucidation can be problematic or in cognitively impaired patients. For example, in the long-term care setting, there are high rates of both asymptomatic bacteriuria (up to 50%) and cognitive impairment.

Thus, the present invention features systems, devices, and methods tailored for diagnosing urinary tract infections (UTIs) in various patient demographics, with a particular emphasis on geriatric and pediatric populations. These innovative solutions address the diagnostic challenges encountered in patients who face difficulties in communicating symptoms, such as those with dementia.

### BRIEF SUMMARY OF THE INVENTION

It is an objective of the present invention to provide systems, devices, and methods that allow for the diagnosis of a urinary tract infection, as specified in the independent claims. Embodiments of the invention are given in the dependent claims. Embodiments of the present invention can be freely combined with each other if they are not mutually exclusive.

Soluble infection-associated biomarkers can be detected in urine, and studies have demonstrated the association of these urinary biomarkers with the presence of a clinically diagnosed UTI. Using such biomarkers, individually or in combination, provides strong evidence of immune response to uropathogens in the urinary tract at the time of urine collection. In an unpublished pilot study (n=100), the Inventors' evaluated five candidate urine biomarkers [neutrophil gelatinase-associated lipocalin (NGAL), interleukins 8, 6, and 1β (IL-8, IL-6, and IL1-β), and matrix metalloproteinase 9 (MMP-9)], and found that three showed a promising correlation with uropathogen detection by M-PCR and SUC in patients symptomatic for UTI: NGAL and IL-8 had good sensitivity and specificity while IL-1β had very high sensitivity (FIG. 13).

In some embodiments, the present invention features a detection system for detecting a urinary tract infection (UTI) in a subject having or suspected of having a urinary tract infection, the system comprising: a) a sample receiving zone to receive a portion of a urine sample from the subject; and b) at least two detection zones for detecting at least two biomarker proteins selected from a group consisting of neutrophil gelatinase-associated lipocalin (NGAL), interleukin-1β (IL-1β), and IL-8. In some embodiments, the detection of at least two biomarker proteins above a reference level is indicative of a UTI.

In some embodiments, the present invention features a method of detecting a urinary tract infection (UTI) in a subject having or suspected of having a urinary tract infection, said method comprising: introducing a portion of a urine sample derived from the patient to a detection system comprising: a) a sample receiving zone to receive the portion of the urine sample from the subject; and b) at least two detection zones for detecting at least two biomarker proteins selected from a group consisting of neutrophil gelatinase-associated lipocalin (NGAL), interleukin-1β (IL-1β), and IL-8. In some embodiments, the detection of at least two biomarker proteins above a reference level is indicative of a UTI.

In some embodiments, the detection system is designed for subjects experiencing atypical symptoms or for pediatric subjects or geriatric subjects. The system may be a biochip, a test strip, a microtiter plate, or a microfluidic plate. Each detection zone within the system may comprise antibodies for binding the biomarker proteins, which are detected using methods such as a lateral flow assay or an ELISA. In some embodiments, the reference levels for the biomarkers are approximately 38 ng/ml for NGAL, 20 pg/mL for IL-8, and 12 pg/mL for IL-1β. Additionally, the detection system includes one or more sensors operatively coupled to the detection zones. These sensors are configured to detect changes in the detection zones in response to the sample, generate a signal based on these changes, and transmit the signal to a computing device. The change in the detection zones could be a change in color, fluorescence, presence of a protein, electrical current, or a combination thereof. The system also comprises a control zone configured to bind a control protein.

In some embodiments, the present invention features an in vitro method comprising obtaining or having obtained a urine sample from a subject having or suspected of having a urinary tract infection and detecting levels of at least two biomarker proteins selected from a group consisting of neutrophil gelatinase-associated lipocalin (NGAL), interleukin-1β (IL-1β), and IL-8. In some embodiments, if the level of at least two biomarker proteins are higher than a reference level, then the subject has a urinary tract infection.

One of the unique and inventive technical features of the present invention is the use of three UTI-associated biomarkers, including neutrophil gelatinase-associated lipocalin (NGAL), interleukin-8 (IL-8), and interleukin-1-beta (IL-1β). Without wishing to limit the invention to any theory or mechanism, it is believed that the technical feature of the present invention advantageously provides for higher sensitivity and specificity for distinguishing a UTI from asymptomatic bacteriuria. None of the presently known prior references or works have the unique inventive technical feature of the present invention.

Moreover, the prior references teach away from the present invention. For instance, many biomarkers currently in use or under consideration exhibit systemic activation, indicating activity wherever there is an infection. To isolate biomarkers specifically indicative of a UTI, the Inventors conducted extensive testing on two distinct populations: those exhibiting UTI symptoms and those asymptomatic. This rigorous analysis encompassed a wide array of biomarkers, ultimately leading to the identification of the three biomarkers (along with their respective thresholds) that exhibited the highest levels of sensitivity, specificity, positive predictive value, negative predictive value, and accuracy.

Additionally, current methods for diagnosing UTIs, such as dipstick tests to detect leukocytes in urine, often yield high false positive results in elderly patients and are less accurate for atypical symptoms, young children, and the elderly compared to young adults. Additionally, dipsticks are more accurate in men than women and can be influenced by menstrual status. Standard urine cultures (SUC) are another common method but are time-consuming, taking up to 3-5 days for bacterial identification and antimicrobial susceptibility testing. SUC also has inherent limitations, including poor sensitivity for detecting slow-growing, fastidious, or non-aerobic microorganisms, and a bias towards fast-growing gram-negative aerobic organisms. Furthermore, the use of SUC can contribute to bacterial resistance when broad-spectrum antibiotics are prescribed as a precautionary measure.

Furthermore, the inventive technical feature of the present invention contributed to a surprising result. For example, the present invention surprisingly has the ability to detect proteins at picogram levels, using fluorescent probes and an external device for probe detection. Following this, the critical hurdles of ensuring clinical validity and establishing the predictive capability of the results in detecting UTIs were effectively addressed.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, and 1C show box plots of NGAL (FIG. 1A), IL-8 (FIG. 1B), and IL-1β (FIG. 1C) biomarker levels in ng/ml (FIG. 1A) or pg/mL (FIGS. 1B and 1C) are shown for different microbial density categories based on M-PCR quantitative data (left) and SUC quantitative data (right). Biomarker concentrations from individual urine specimens are indicated by open circles. Whiskers extend from the minimum to the maximum detected biomarker concentrations, biomarker medians for each microbial density category are marked with a horizontal line and used for statistical analysis, and biomarker means for each microbial density category are marked with a “+”. The black dotted line marks the threshold for NGAL positivity (38 ng/ml; FIG. 1A), IL-8 positivity (20.6 pg/mL; FIG. 1B), and IL-1β positivity (12.4 pg/mL; FIG. 1C). Shading indicates values below the threshold.

FIG. 2 shows box plots of NGAL (ng/ml), IL-8 (pg/mL), and IL-1β (pg/mL) biomarker levels from specimens in which no microbes were detected (n=117 by M-PCR, n=193 by SUC). Values from individual urine specimens are indicated by open circles. Whiskers extend from the minimum to the maximum detected value; means are indicated with a “+”; medians for each microbial density category are marked with a horizontal line and used for statistical analysis.

FIGS. 3A and 3B show biomarker consensus displayed for different microbial densities, and biomarker consensus positivity percentage plotted along the x-axis. Microbial density categories as detected via SUC (FIG. 3A) and M-PCR (FIG. 3B). The number of specimens in each microbial density category and the biomarker consensus percent positivity are labeled at the end of each bar.

FIGS. 4A, 4B, 4C, and 4D show box plots of biomarker levels with microbial detection (FIG. 4A), microbial detection of E. coli cases (FIG. 4B), and detection of non-E. coli microorganisms (FIG. 4C), and by standard urine culture (SUC) and multiplex polymerase chain reaction (M-PCR). FIG. 4D shows box plots of biomarker levels with polymicrobial detection by M-PCR. Detection method: Microbial density categories are along the x-axis. Individual biomarker values measured by enzyme-linked immunosorbent assay (ELISA) are plotted along the y-axis as open circles. Boxes extend from the first to the third quartiles, with whiskers extending to the minimum and maximum values. Within each plot, a solid horizontal line indicates the median value, and a “+” indicates the mean. A dotted line represents the positivity threshold for each biomarker (neutrophil gelatinase-associated lipocalin [NGAL]≥38.0 ng/ml, interleukin 8 [IL-8]≥20.6 pg/ml, and interleukin 1 beta [IL-1β]≥12.4 pg/ml).

FIG. 5 shows Biomarker percent positivity with microbial detection by M-PCR and SUC. Comparison of percent positivity for biomarker consensus, NGAL, IL-8, and IL-1β between specimens in which M-PCR and SUC results were discordant at a microbial density threshold of >10 000 cells/ml or CFUs/ml. Biomarker positivity was defined by previously published thresholds (NGAL≥38.0 ng/ml, IL-8≥20.6 pg/ml, and IL-1β≥12.4 pg/ml). CFU=colony forming unit; IL-1b=interleukin 1 beta; IL-8=interleukin 8; M-PCR=multiplex polymerase chain reaction; NGAL=neutrophil gelatinase-associated lipocalin; SUC=standard urine culture.

FIG. 6 shows Organism detection prevalence. Note: Organisms or organism groups are arranged on the y-axis in descending order of detection prevalence. Bar length along the x-axis represents the percent of M-PCR-positive specimens (microbial density≥10,000 cells/mL for bacteria/bacterial groups or >0 cells/mL for yeasts). The number (n) of detections are shown with labels at the end of each bar.

FIG. 7 shows biomarker consensus percent positivity by microbial identification. Notes: Organisms or organism groups are listed on the y-axis in descending order of detection prevalence. Bar length along the x-axis represents the percentage of positive specimens (microbial density≥10,000 cells/mL for bacteria/bacterial groups or >0 cells/mL for yeasts) that are also positive for biomarker consensus. Labels at the end of each bar show the associated biomarker consensus percentage and the number of biomarker consensus positive specimens out of the total number of specimens positive for that organism (in parenthesis).

FIG. 8 shows biomarker Positivity in M-PCR Positive versus Negative Cases. Note: Bolded values indicate p<0.05.

FIG. 9 shows biomarker Positivity in M-PCR Positive Cases Grouped by Organism and Infection Characteristics. Note: Bolded values indicate p<0.05.

FIG. 10A shows biomarker Positivity in M-PCR Positive Versus Negative Cases.

FIG. 10B shows biomarker Positivity in M-PCR Positive Cases Grouped by Organism and Infection Characteristics.

FIG. 11A-11D show positive Correlation Between Microbial Density by M-PCR and Biomarker Positivity. The probit regression lines demonstrate a significant positive correlation between urine microbial density and biomarker consensus (FIG. 11A), NGAL (FIG. 11B), IL-8 (FIG. 11C), and IL-1β (FIG. 11D) positivity in both symptomatic (black), and asymptomatic (grey) subjects. Each data point indicates the proportion of biomarker positivity (x-axis) for urine specimens at each of the semi-quantitatively reported microbial densities in cells/mL (≤10⁴ 10⁴ to 10⁵, 10⁵ to 10⁶, 10⁶ to 10⁷, 10⁷ to 10⁸, and ≥10⁸ for M-PCR or ≤10⁴, 10⁴ to 10⁵, and ≥10⁵ for SUC) presented along the y-axis. A probit regression analysis line is shown connecting the data points

FIG. 12 shows biomarker Levels are Low in Definitive non-UTIs Regardless of Microbial Detection. Tukey boxplots extending to the 1st and 3rd quartiles with a line at the median indicate the distribution of biomarker (NGAL, IL-8, and IL-1β) levels among each group presented on the x-axis. Biomarker measurements are plotted along the y-axis with each point representing the measurement for a single urine specimen. Groups presented on the x-axis for comparison include “Definitive UTIs” cases (specimens from symptomatic subjects in which microorganisms are detected by both M-PCR and SUC at ≥10,000 cells/mL or CFUs/mL respectively), and “Definitive non-UTI” cases (asymptomatic cohort specimens). The “Definitive non-UTI” cases are further divided by microbial detection category: no microbes, microbes detected by SUC or M-PCR, and microbes detected by both SUC and M-PCR (Dual+)

FIG. 13 shows descriptive Statistics of Biomarker Values for the Definitive UTI and Definitive non-UTI Cohorts Based on Criterion

FIG. 14 shows asymptomatic Microbial Density. The bacterial density for each asymptomatic case as measured by M-PCR, with the median density (horizontal line), at 239,611 and the mean density of 2.8×107 Cells/mL. Each individual dot within the figure represents a distinct specimen, highlighting the diverse range of densities observed within the dataset. This figure visually captures the variability and trends in bacterial densities within asymptomatic bacteriuria cases.

FIG. 15 shows non-limiting examples of systems described herein.

FIG. 16 shows Common Clinical Signs and Symptoms Spectrum in Women. Women's clinical signs and symptoms frequently overlap between urologic diagnoses, including urinary tract infection, interstitial cystitis/bladder pain syndrome, overactive bladder, urinary incontinence, asymptomatic bacteriuria, and others (not shown). Any of the indicated symptoms may be present for each condition, but they do not all have to be present.

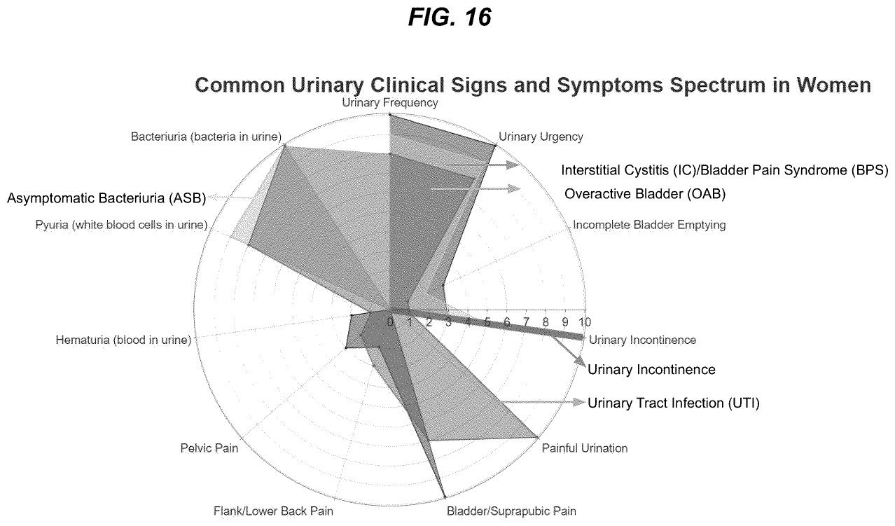

### DETAILED DESCRIPTION OF THE INVENTION

Unless otherwise explained, all technical and scientific terms used herein have the same meaning as commonly understood by one of ordinary skill in the art to which a disclosed invention belongs. All publications, patent applications, patents, and other references mentioned herein are incorporated by reference in their entirety for all purposes. In case of conflict, the present specification, including explanations of terms, will control.

The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder, or condition described herein. In certain instances, the term patient refers to a human.

As used herein, a “urinary tract infection (UTI)” refers to an infection in any part of the urinary system. Typical Signs of a UTI include a burning sensation when urinating, frequent urination even with minimal output, an urgent need to urinate, cloudy or strong-smelling urine, and the presence of blood in the urine (hematuria). Additionally, pelvic pain or pressure, especially in women, may indicate a UTI. Atypical Signs of a UTI encompass symptoms such as increased falls, particularly in older adults who may experience confusion, agitation, or delirium due to the infection. Lower back pain may suggest the infection has reached the kidneys, while fever, chills, nausea, vomiting, fatigue, confusion, weakness, muscle aches, and incontinence may also occur. These symptoms can disrupt daily living, leading to disrupted sleep, reduced physical activity, decreased concentration, and emotional distress due to persistent pain and inconvenience. Below is a comprehensive and detailed description of the aforementioned symptoms.

Typical Signs of a UTI include:

| | |
|---|---|
| Burning sensation | One of the most common symptoms. |
| (when urinating): | |
| Frequent urination: | Feeling the urge to urinate more often than usual. |
| Urgent need to urinate: | Experiencing a strong and persistent urge to urinate. |
| Cloudy urine: | Urine that appears cloudy. |
| Strong-smelling urine: | Urine with a strong or unpleasant odor. |
| Blood in urine: | Urine that appears red, bright pink, or cola-colored, indicating the presence |
| | of blood (hematuria). |
| Pelvic pain: | Pain or pressure in the lower abdomen or pelvic area, especially in women. |

Atypical Signs of a UTI may include:

| | |
|---|---|
| Increased falls: | UTIs can lead to confusion and delirium, particularly in older adults, |
| | increasing the risk of falls. |
| Lower back pain: | Discomfort in the lower back, possibly indicating a kidney infection. |
| Fever and chills: | Elevated temperature and chills may occur if the infection spreads to the |
| | kidneys (pyelonephritis). |
| Nausea and vomiting: | Symptoms that can accompany a kidney infection. |
| Fatigue: | Feeling unusually tired or fatigued. |
| Confusion or agitation: | Especially in older adults, UTIs can cause mental changes. |
| Weakness: | A general feeling of weakness or malaise. |
| Muscle aches: | Unexplained muscle pains. |
| Incontinence: | Inability to control the bladder, resulting in accidental urine leakage. |

Alterations in Daily Living Due to a UTI may include:

| | |
|---|---|
| Disrupted Sleep: | Frequent urination interrupts sleep patterns, leading to fatigue. |
| Reduced Physical | Pain and discomfort may limit physical activities or exercise. |
| Activity: | |
| Decreased | Discomfort and frequent bathroom trips can affect concentration. |
| Concentration: | |
| Emotional Distress: | Persistent pain and inconvenience can lead to increased stress and anxiety. |

As used herein, “acute cystitis” indicates the sudden onset of symptoms consistent with UTI, confirmed by the presence of uropathogens in urine samples (typically via standard urine culture, SUC).

As used herein, an “uncomplicated UTI (uUTI)” refers to a urinary tract infection occurring in a healthy, non-pregnant adult patient with no known UTI-associated risk factor.

As used herein, a “complicated UTI (cUTI)” refers to a urinary tract infection in a patient with one or more risk factors that heighten the risk of serious adverse outcomes or potentially reduce the effectiveness of therapy. These risk factors include anatomical and functional abnormalities of the urinary tract, physical obstructions such as stones and calculi, catheterization, an immunocompromised status, male sex, advanced age, diabetes mellitus, the presence of antimicrobial-resistant organisms, or atypical organisms.

As used herein, a “recurrent UTI (rUTI)” refers to two symptomatic episodes of acute cystitis within six months or three episodes within one year, with confirmation of uropathogens from urine samples (typically via standard urine culture, SUC).

As used herein, “asymptomatic Bacteriuria (ASB)” is characterized by the presence of bacteria in the urine of a subject without any signs of illness or symptoms.

As used herein, a “definitive UTI” defines subjects who were symptomatic, with a diagnosis of UTI in a urology specialty setting, and who had positive microbe detection

As used herein, a “definitive non-UTI” defines asymptomatic subjects either with microbes detected in the urine (asymptomatic bacteriuria) or without.

“Signal producing component” refers to any substance capable of reacting with another assay reagent or with the biomarker(s) to produce a reaction product or signal that indicates the presence of the biomarker(s) and that is detectable by visual or instrumental means. “Signal production system”, as used herein, refers to the group of assay reagents that are needed to produce the desired reaction product or signal.

“Observable signal” as used herein refers to a signal produced in the claimed devices and methods that is detectable by visual inspection. Generally, observable signals indicating the presence or absence of a biomarker in a sample may be evident of their own accord, e.g., plus or minus signs or particularly shaped symbols, or may be evident through the comparison with a panel such as a color indicator panel.

As used herein the terms “upstream” and “downstream” refer to the direction of a sample flow subsequent to contact of the sample with a representative device of the present disclosure, wherein, under normal operating conditions, the fluid sample flow direction runs from an upstream position to a downstream position. For example, when fluid sample is initially contacted with the sample receiving zone, the fluid sample then flows downstream through the conjugating zone and so forth.

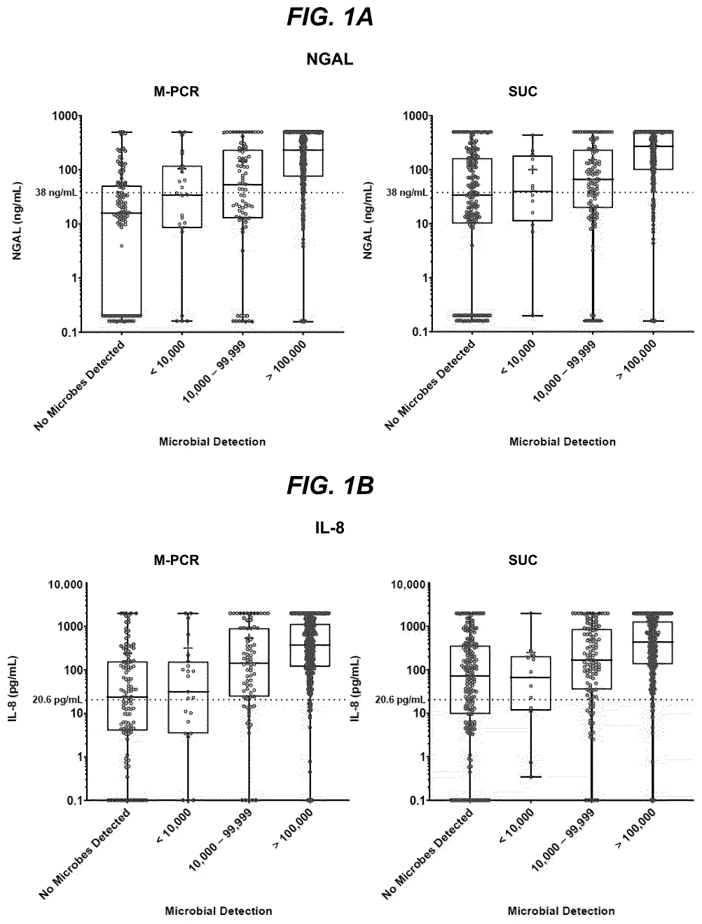

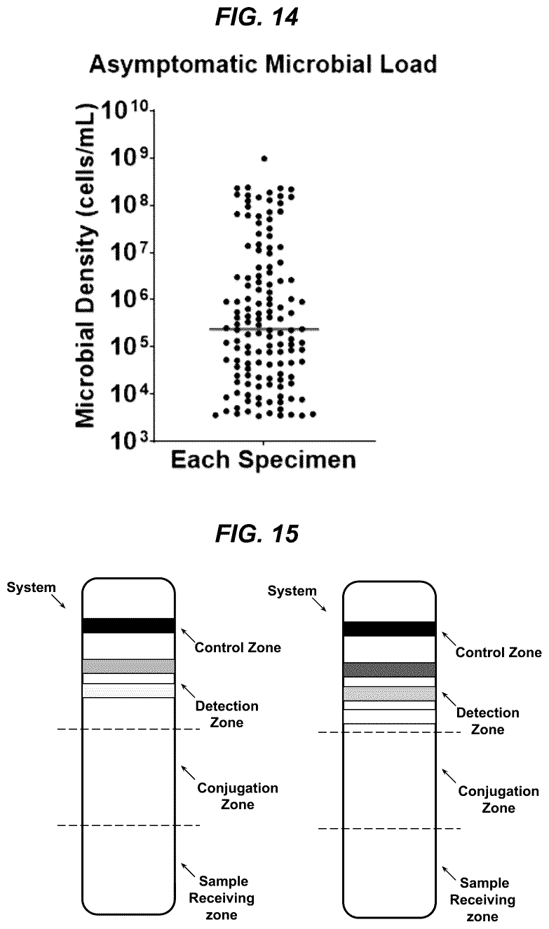

Referring now to FIGS. 1-15, the present invention features point-of-care methods, systems, and devices designed to indicate a patient's immune response to pathogens in the urinary tract, such as those associated with urinary tract infections. The methods, systems, and devices herein focus on the detection of UTI-associated biomarkers, including neutrophil gelatinase-associated lipocalin (NGAL), interleukin-8 (IL-8), and interleukin-1-beta (IL-1β). These biomarkers, when detected above specific thresholds in urine, collectively serve as strong indicators of the patient's immune response, leading to a more accurate diagnosis of UTI and determining the necessity for follow-up testing

The present invention may feature an in vitro method comprising obtaining or having obtained a urine sample from a subject having or suspected of having a urinary tract infection and detecting levels of at least two biomarker proteins selected from a group consisting of neutrophil gelatinase-associated lipocalin (NGAL), interleukin-1β (IL-1β), and IL-8. In some embodiments, if the level of at least two biomarker proteins is higher than a reference level for said biomarker protein, then the subject has a urinary tract infection. In some embodiments, the method may comprise obtaining or having obtained a urine sample from a subject having or suspected of having a urinary tract infection and detecting levels of three biomarker proteins selected from a group consisting of neutrophil gelatinase-associated lipocalin (NGAL), interleukin-1β (IL-1β), and IL-8. In other embodiments, the method comprises obtaining or having obtained a urine sample from a subject having or suspected of having a urinary tract infection and detecting levels of at least two biomarker proteins selected from a group consisting essentially of neutrophil gelatinase-associated lipocalin (NGAL), interleukin-1β (IL-1β), and IL-8. In further embodiments, the method comprises obtaining or having obtained a urine sample from a subject having or suspected of having a urinary tract infection and detecting levels of three biomarker proteins selected from a group consisting essentially of neutrophil gelatinase-associated lipocalin (NGAL), interleukin-1β (IL-1β), and IL-8. Again, In some embodiments, if the level of at least two biomarker proteins is higher than a reference level for said biomarker protein, then the subject has a urinary tract infection.

The present invention may also feature an in vitro method for distinguishing between asymptomatic bacteriuria and a urinary tract infection (UTI) in a subject having or suspected of having a urinary tract infection. For example, the method may comprise obtaining or having obtained a urine sample from the subject and detecting levels of at least two biomarker proteins selected from a group consisting of neutrophil gelatinase-associated lipocalin (NGAL), interleukin-1β (IL-1β), and IL-8. In some embodiments, if the level of at least two biomarker proteins is higher than a reference level for said biomarker protein, then the subject has a urinary tract infection. In other embodiments, the method may comprise obtaining or having obtained a urine sample from the subject and detecting levels of three biomarker proteins selected from a group consisting of neutrophil gelatinase-associated lipocalin (NGAL), interleukin-1β (IL-1β), and IL-8. In some embodiments, if the level of at least two biomarker proteins is higher than a reference level for said biomarker protein, then the subject has a urinary tract infection. In further embodiments, the method comprises obtaining or having obtained a urine sample from the subject and detecting levels of at least two biomarker proteins selected from a group consisting essentially of neutrophil gelatinase-associated lipocalin (NGAL), interleukin-1β (IL-1β), and IL-8. In some embodiments, if the level of at least two biomarker proteins is higher than a reference level for said biomarker protein, then the subject has a urinary tract infection.

The present invention may further feature an in vitro method for the diagnosis of a urinary tract infection (UTI) in a subject. In some embodiments, the method comprises a) providing a urine sample from the subject and b) detecting the levels of at least two biomarker proteins selected from a group consisting (or consisting essentially) of neutrophil gelatinase-associated lipocalin (NGAL), interleukin-1β (IL-1β), and IL-8 in the urine sample. In some embodiments, a higher level of at least two of the biomarkers in the urine sample as determined in step b) compared to a reference level is indicative of a UTI in the subject. In other embodiments, the method comprises a) providing a urine sample from the subject and b) detecting the levels of three biomarker proteins selected from a group consisting (or consisting essentially) of neutrophil gelatinase-associated lipocalin (NGAL), interleukin-1β (IL-1β), and IL-8 in the urine sample. In some embodiments, a higher level of at least two of the biomarkers in the urine sample as determined in step b) compared to a reference level is indicative of a UTI in the subject.

In certain embodiments, the present invention may further feature an in vitro method for the diagnosis of a urinary tract infection (UTI) in a subject. In some embodiments, the method comprises a) obtaining or having obtained a urine sample from the subject and b) detecting or having detected the levels of at least two biomarker proteins selected from a group consisting (or consisting essentially) of neutrophil gelatinase-associated lipocalin (NGAL), interleukin-1β (IL-1β), and IL-8 in the urine sample. In some embodiments, a higher level of at least two of the biomarkers in the urine sample as determined in step b) compared to a reference level is indicative of a UTI in the subject. In other embodiments, the method comprises a) obtaining or having obtained a urine sample from the subject and b) detecting or having detected the levels of three biomarker proteins selected from a group consisting (or consisting essentially) of neutrophil gelatinase-associated lipocalin (NGAL), interleukin-1β (IL-1β), and IL-8 in the urine sample. In some embodiments, a higher level of at least two of the biomarkers in the urine sample as determined in step b) compared to a reference level is indicative of a UTI in the subject.

In some embodiments, the methods described herein may be applied to adult subjects. Alternatively, the methods may be utilized for pediatric subjects or geriatric subjects. In specific instances, the methods are adapted for geriatric patients, defined as individuals over 65 years old, as well as Alzheimer's or dementia patients. Moreover, in certain embodiments, the methods herein are particularly suitable for subjects who face challenges in communicating, as observed in geriatric and pediatric patients. This is especially pronounced in patients with dementia, who frequently exhibit atypical symptoms such as alterations in behavior or cognition.

In some embodiments, the method involves utilizing a voided urine sample. Alternatively, the method may utilize a catheterized urine sample. Additionally, in certain instances, the method involves obtaining the urine sample from an undergarment or a diaper. However, the present invention is not limited to the aforementioned methods of collecting urine and may include any other methods known in the art. For example, urine samples may be obtained through midstream collection, first stream collection, catheterization, suprapubic aspiration, a urine collection bag, or as a random urine sample.

Urine samples can be obtained through various methods, both non-invasive and invasive. Non-invasive methods include midstream clean-catch collection, commonly used for routine urinalysis and cultures, which involves cleaning the genital area and collecting urine midstream to reduce contamination. Invasive methods may include catheterization, where a catheter is inserted into the bladder to collect urine, and suprapubic aspiration, involving a needle inserted through the abdominal wall into the bladder, both used for sterile samples, particularly when patients cannot void naturally or when other methods are not feasible.

Pediatric methods may involve a urine collection bag, an adhesive bag placed over the genital area to collect urine from infants and young children, or a pediatric urine collection pad, an absorbent pad placed in the diaper from which urine is extracted for analysis. Specialized methods for females include a clean-catch bag, a special bag fitting over the genital area to catch urine when midstream collection is challenging, and a urine collection hat/pan, a hat-shaped container placed under the toilet seat used in hospital or clinical settings for convenient collection.

Urine diversion devices include a urostomy bag, used for patients with a urostomy to collect urine directly from the stoma, and an external condom catheter, used for males, fitting over the penis like a condom and draining urine into a bag. Non-invasive alternatives to indwelling catheters include home collection devices or kits, pre-packaged for collecting urine samples at home, often used for convenience and privacy in routine testing.

However, the present invention is not limited to the aforementioned methods of collecting urine and may include any other methods known in the art.

In some embodiments, the reference level for the NGAL protein biomarker is about 38.0 ng/ml. In some embodiments, the reference level for the NGAL protein biomarker is about 40 ng/ml. In some embodiments, the reference level for the IL-8 protein biomarker is about 20.6 pg/mL. In some embodiments, the reference level for the IL-8 protein biomarker is about 20 pg/mL. In some embodiments, the reference level for the IL-8 protein biomarker is about 25 pg/mL. In some embodiments, the reference level for the IL-1β protein biomarker is about 12.4 pg/mL. In some embodiments, the reference level for the IL-1β protein biomarker is about 12 pg/mL. In some embodiments, the reference level for the IL-1β protein biomarker is about 15 pg/mL. Without wishing to limit the present invention to any theory of mechanism, it is believed that a biomarker protein at or above the aforementioned reference levels indicates a positive result (e.g., the biomarker is elevated).

In some embodiments, the method involves detecting protein biomarkers using various techniques such as Western blot, dot blot, Enzyme-linked immunosorbent assay (ELISA), or lateral flow assay. Additionally, biomarker detection may involve the use of one or more antibodies, e.g., for ELISA and lateral flow assays. In certain embodiments, a sandwich ELISA is utilized. Alternatively, in some embodiments, protein biomarkers may be detected via mass spectrometry, metagenomic next-generation sequencing (mNGS), or microfluidics. The present invention is not limited to the aforementioned methods and may utilize any method known in the art capable of detecting protein biomarkers at low levels (e.g., in nanograms or picograms).

In some embodiments, the methods described herein may further comprise detecting one or a combination of nitrites, carbohydrates, white blood cells (WBCs), or red blood cells (RBCs). Moreover, these methods are capable of detecting fragments of WBCs or RBCs as well.

The present invention may also feature a detection system comprising a sample receiving zone to receive a sample from a subject having or suspected of having a urinary tract infection and at least two detection zones for detecting at least two biomarker proteins selected from a group consisting (or consisting essentially) of neutrophil gelatinase-associated lipocalin (NGAL), interleukin-1β (IL-1β), and IL-8. In other embodiments, the detection system comprises a sample receiving zone to receive a sample from a subject having or suspected of having a urinary tract infection and three detection zones for detecting biomarker proteins selected from a group consisting (or consisting essentially) of neutrophil gelatinase-associated lipocalin (NGAL), interleukin-1β (IL-1β), and IL-8. In certain embodiments, the detection systems herein exhibit the capability to detect protein biomarkers at low levels, such as in nanograms or picograms. Moreover, in specific instances, if the protein biomarker increases over a predetermined reference level (as discussed above), the sample is deemed positive for said biomarker protein. Notably, in some embodiments, samples testing positive for two out of three or all three biomarkers indicate the presence of a urinary tract infection (UTI) in the subject.

In certain embodiments, the system described herein may take the form of a biochip, a test strip, a microtiter plate, a microfluidic plate, or similar devices. In some embodiments, the system is in the form of a dipstick assay device. In some embodiments, biomarker proteins are detected utilizing methods such as a lateral flow assay or an Enzyme-linked immunosorbent assay (ELISA). In some embodiments, each of the detection zones comprises antibodies for binding the biomarker proteins. In other embodiments, each of the detection zones comprises antibodies for binding individual biomarker proteins.

The present invention may also feature a detection system for detecting a urinary tract infection (UTI) in a subject having or suspected of having a UTI. In some embodiments, the system is configured to detect the presence of a combination of neutrophil gelatinase-associated lipocalin (NGAL), interleukin-1β (IL-1β), and IL-8 in a urine sample from the subject. In some embodiments, the detection of at least two of NGAL, IL-1β, and IL-8 above a reference level is indicative of a UTI. In other embodiments, the system is configured to detect the presence of neutrophil gelatinase-associated lipocalin (NGAL), interleukin-1β (IL-1β), and IL-8 in a urine sample from the subject, wherein detection of at least two of NGAL, IL-1β, and IL-8 above a reference level is indicative of a UTI.

In some embodiments, the present invention features a kit comprising a detection system as described herein.

The present invention may further feature a system for detecting a urinary tract infection (UTI) in a subject, e.g., a subject having or suspected of having a UTI. In some embodiments, the system comprises a) a sample receiving zone to which a urine sample from the subject is added, b) a conjugating zone positioned downstream from the sample receiving zone, said conjugating zone comprising at least two mobilizable signal-producing components, wherein each mobilizable signal producing component binds to one of the biomarkers selected from a group consisting (consisting essentially) of neutrophil gelatinase-associated lipocalin (NGAL), IL-1β, and IL-8. c) at least two detection zones positioned downstream from the conjugating zone, wherein each detection zone comprises a component that binds each biomarker conjugated to a mobilizable signal-producing component and d) a control zone positioned downstream from the detection zone, the control zone comprising an immobilized component that binds with the labeled control reagent.

In other embodiments, the system comprises a) a sample receiving zone to which a urine sample from the subject is added, b) a conjugating zone positioned downstream from the sample receiving zone, said conjugating zone comprising at least three mobilizable signal-producing components, wherein each mobilizable signal producing component binds to one of the biomarkers selected from a group consisting (consisting essentially) of neutrophil gelatinase-associated lipocalin (NGAL), IL-1β, and IL-8. c) at least three detection zones positioned downstream from the conjugating zone, wherein each detection zone comprises a component that binds each biomarker conjugated to a mobilizable signal-producing component and d) a control zone positioned downstream from the detection zone, the control zone comprising an immobilized component that binds with the labeled control reagent.

Non-limiting examples of the mobilizable signal-producing component include a chemiluminescent agent, a particulate label, a colorimetric agent, an energy transfer agent, an enzyme, a fluorescent agent, and a radioisotope. In some embodiments, the mobilizable signal-producing component is a fluorescent agent such as a fluorescent probe, e.g., a fluorescent probe conjugated to an antibody.

In some embodiments, each detection zone comprises an antibody that binds the biomarker. In some embodiments, the immobilized component in the control zone comprises an antibody. For example, the antibody in the control zone may be specific for a labeled control reagent.

In certain embodiments, the aforementioned systems may take the form of a biochip, a test strip, a microtiter plate, a microfluidic plate, or similar devices. In some embodiments, the system is in the form of a dipstick assay device. In some embodiments, biomarker proteins are detected utilizing methods such as a lateral flow assay or an Enzyme-linked immunosorbent assay (ELISA). In some embodiments, each of the detection zones comprises antibodies for binding the biomarker proteins. In other embodiments, each of the detection zones comprises antibodies for binding individual biomarker proteins.

In some embodiments, the present invention features a lateral flow system for detecting a urinary tract infection (UTI) in a patient, said system comprises a lateral flow assay for a urine sample, wherein the lateral flow assay is configured to detect the presence of at least two of neutrophil gelatinase-associated lipocalin (NGAL), IL-1β, and IL-8 in the urine sample, wherein detection of at least two of NGAL, IL-1β, and IL-8 above a reference level is indicative of a UTI. In other embodiments, the present invention features a lateral flow system for detecting a urinary tract infection (UTI) in a patient, said system comprises a lateral flow assay for a urine sample, wherein the lateral flow assay is configured to detect the presence of neutrophil gelatinase-associated lipocalin (NGAL), IL-1β, and IL-8 in the urine sample, wherein detection of at least two of NGAL, IL-1β, and IL-8 above a reference level is indicative of a UTI. In some embodiments, the lateral flow system is able to distinguish asymptomatic bacteriuria and a urinary tract infection (UTI) in the patient.

In some embodiments, the present invention features a lateral flow system for distinguishing between an asymptomatic bacteriuria and a urinary tract infection (UTI) in a patient, said system comprises a lateral flow assay for a urine sample, wherein the lateral flow assay is configured to detect the presence of at least two of neutrophil gelatinase-associated lipocalin (NGAL) and IL-1β, and IL-8 in the urine sample, wherein detection of at least two of neutrophil gelatinase-associated lipocalin (NGAL), IL-1β, and IL-8 is indicative of a UTI. In other embodiments, the present invention features a lateral flow system for distinguishing between an asymptomatic bacteriuria and a urinary tract infection (UTI) in a patient, said system comprising: a lateral flow assay for a urine sample, wherein the lateral flow assay is configured to detect the presence of neutrophil gelatinase-associated lipocalin (NGAL) and IL-1β, and IL-8 in the urine sample, wherein detection of at least two of neutrophil gelatinase-associated lipocalin (NGAL), IL-1β, and IL-8 is indicative of a UTI.

The present invention may also feature a method of measuring the levels of biomarker proteins in a biological sample from a subject having or suspected of having a urinary tract infection (UTI). In some embodiments, the method comprising a) measuring or having measured levels of at least two biomarker proteins selected from a group consisting of neutrophil gelatinase-associated lipocalin (NGAL), interleukin-1β (IL-1β), and IL-8, b) detecting or having detected a changed level of NGAL, IL-1β, and IL-8 relative to a reference level of NGAL, IL-1β, and IL-8; and administering or having administered a treatment for a UTI when an increased level of at least two biomarkers are detected.

In some embodiments, the present invention may comprise one or more sensors operatively coupled to the detection zones. The one or more sensors may be configured to detect a change in the detection zones in response to the sample, generate a signal based on the change in the detection zones, and transmit the signal to a computing device. In some embodiments, the change in the detection zones may be a change in color, fluorescence, presence of a protein, electrical current, or a combination thereof.

In some embodiments, the one or more sensors may be communicatively coupled to a computing device. The one or more sensors may be coupled to the computing device by a wired connection, a wireless connection (e.g. Bluetooth®, WiFi, wireless local area network (WLAN), wide area network (WAN), etc.), or a combination thereof. In some embodiments, the one or more sensors may be coupled to an external computing device. In other embodiments, the one or more sensors may be integrated with the computing device in a single device.

In some embodiments, the computing device may comprise a processor configured to execute computer-readable instructions. The computing device may further comprise a memory component operatively coupled to the processor, comprising computer-readable instructions. The computer-readable instructions may comprise accepting the signal from the one or more sensors. The computer-readable instructions may further comprise measuring the signal. The process of measuring the signal is dependent on the type of sensors that are used. For example, if the sensors are configured to detect a change in color, measuring the signal may comprise measuring absorption, reflectance, or a combination thereof. In some embodiments, the computer-readable instructions may further comprise determining, based on the measured signal, whether the change in the detection zones exceeds a certain threshold. For example, the computing device may determine whether the absorption, the reflectance, the amount of a certain particle, the electrical current, or a combination thereof exceeds a certain threshold. In some embodiments, the computing device may further comprise a display component operatively coupled to the processor. In some embodiments, the memory component may further comprise computer-readable instructions for displaying whether or not the change in the detection zones exceeded the threshold.

In some embodiments, the one or more sensors may comprise one or more imaging components configured to image the detection zones of the detection system of the present invention. In some embodiments, the one or more imaging components may be disposed optically in-line with the detection zones. In other embodiments, the one or more imaging components may be disposed in a movable component configured to be positioned optically in-line with the detection zones (e.g. in a handheld device).

In some embodiments, each imaging component of the one or more imaging components may comprise a light source configured to illuminate the detection zones with light at one or more wavelengths. In some embodiments, the light may be configured to highlight the change in the detection zones. For example, the light may be configured to heighten the fluorescence of a change in color in the detection zones. In another non-limiting example, the light may be configured to excite one or more particles of a protein such that they are more easily detectable.

In some embodiments, each imaging component of the one or more imaging components may further comprise an image processing component configured to capture one or more images of the detection zones. In some embodiments, the image processing component may comprise a lens. The image processing component may further comprise one or more optical elements (e.g. filters, mirrors, windows, prisms, polarizers, beamsplitters, wave plates, fiber optics, retroreflectors, optical flats, or a combination thereof). In some embodiments, the one or more imaging components may comprise a camera configured to capture normal images, infrared images, ultraviolet images, and/or any other form of image.

In some embodiments, the one or more sensors may comprise imaging sensors, electrochemical sensors, chemical sensors, electrical sensors, fluorescence sensors, or a combination thereof. In some embodiments, the one or more sensors may comprise a sensor for each detection zone. In some embodiments, each sensor may be configured to measure changes in a plurality of detection zones. In some embodiments, each detection zone may have its changes measured by a plurality of sensors.

### Example 1

The following is a non-limiting example of the present invention. It is to be understood that said example is not intended to limit the present invention in any way. Equivalents or substitutes are within the scope of the present invention.

The literature lacks consensus on the minimum microbial density required for diagnosing urinary tract infections (UTIs). As described in this example, the present invention categorized the microbial densities of urine specimens from symptomatic UTI patients aged≥60 years and correlated them with detected levels of the immune response biomarkers neutrophil gelatinase-associated lipocalin (NGAL), interleukin-8 (IL-8), and interleukin-1-beta (IL-1β). The objective was to identify the microbial densities associated with significant elevation of these biomarkers in order to determine an optimal threshold for diagnosing symptomatic UTIs. Biobanked midstream voided urine samples were analyzed for microbial identification and quantification using standard urine culture (SUC) and multiplex-polymerase chain reaction (M-PCR) testing, while NGAL, IL-8, and IL-1β levels were measured via enzyme-linked immunosorbent assay (ELISA). NGAL, IL-8, and IL-1β levels were all significantly elevated at microbial densities≥10,000 cells/mL when measured via M-PCR (p<0.0069) or equivalent colony-forming units (CFUs)/mL via SUC (p<0.0104) compared to samples with no detectable microbes. With both PCR and SUC, a consensus of two or more elevated biomarkers correlated well with microbial densities>10,000 cells/mL or CFU/mL, respectively. The association between ≥10,000 cells and CFU per mL with elevated biomarkers in symptomatic patients suggests that this lower threshold may be more suitable than 100,000 CFU/mL for diagnosing UTIs.

In published guidelines, there is a pressing need for greater consensus regarding the minimum microbial threshold for diagnosing a urinary tract infection (UTI). The existing literature presents conflicting information on the commonly used diagnostic threshold of 100,000 colony-forming units (CFUs)/mL, with surprisingly scant and dated evidence to support it. The confusion regarding a minimum threshold has led to uncertainty amongst clinicians, which can lead to increased use of empiric therapy or under treatment of UTIs caused by lower microbial densities. Ensuring accurate diagnosis of symptomatic patients with complicated urinary tract infections (cUTIs) is crucial because these patients often possess one or more risk factors that can lead to treatment failure, adverse clinical outcomes, and/or severe complications. There is a gap in contemporary studies evaluating the correlation between rising microbial density and the presence or absence of a UTI.

Due to the limitations of standard urine culture (SUC), which make it a flawed gold standard test for diagnosis, it is important to identify cases that have a very high likelihood of being true UTIs in any study evaluating diagnostic tests and thresholds. As described herein, true UTI cases were defined as specimens from symptomatic patients that had a clinical diagnosis in a specialist setting for UTI, had a positive identification of known uropathogens via multiplex polymerase chain reaction test (M-PCR) or SUC, and had elevated urine biomarkers that were documented to show inflammation of the urinary tract and have high specificity for UTI.

The uroepithelium and resident innate immune cells in the bladder quickly protect against microbial threats by first identifying microbial patterns and triggering an immune response that produces antimicrobial peptides and pro-inflammatory cytokines. The biomarkers used herein [neutrophil gelatinase-associated lipocalin (NGAL), interleukin-8 (IL-8), and interleukin-1-beta (IL-1β)] are essential components of the constitutive immune response in the urinary tract and have been studied in association with UTIs. One previous study discovered that NGAL, IL-8, and IL-1β have sensitivities of 82.6%, 91.2%, and 69.8% with specificities of 90.8%, 76.8%, and 96.9%, respectively. A consensus of two or more of these biomarkers meeting the threshold of positivity yielded a sensitivity of 84.0% and a specificity of 91.2%.

Using these biomarkers plus a UTI diagnosis in a specialty setting to identify UTI cases in patients 60 years of age and older, biomarker levels were evaluated at different microbial densities using both M-PCR and SUC. The aim was to determine if there was a particular minimal microbial density threshold at which the biomarkers in these cases were significantly elevated, indicating that below this density, a UTI was unlikely. Thus, it would be useful to assess if there is a threshold over which a significant majority of UTIs are present in symptomatic patients. Several different categories of microbial densities and their relationship to levels of inflammatory biomarkers were examined in order to determine if a threshold significantly lower than 100,000 CFU/mL is more appropriate as a general benchmark to diagnose UTIs. Evidence of a lower threshold would call into question the long-standing practice of considering lower densities as incidental findings, which has the potential to lead to the underdiagnosis of patients showing symptoms of a UTI.

Study Design: This study utilized banked urine specimens from patients presenting at urology clinics in 39 U.S. states and assigned ICD-10-CM codes consistent with UTI, and compared urine inflammation biomarker results to microbial quantification results. These ICD-10-CM codes are assigned in the urology specialty setting based on the clinical presentation of the patient and are routinely transmitted to the lab with the diagnostic test order and urine specimen. Only specimens sent for UTI diagnostic testing that also had ICD-10-CM codes that indicated either a UTI or a UTI-related concern were selected for this study. There were 583 specimens included from consecutive eligible subjects which were collected between 17 Jan. 2023 and 24 Apr. 2023. Full inclusion and exclusion criteria are described in Table 2.

TABLE 2
Inclusion and Exclusion criteria

| Inclusion | Exclusion |
|---|---|
| At least 60 years of age | Failure to meet all |
| Male or Female sex | inclusion criteria |
| (No predetermined quotas or ratios for participants) | |
| Presenting to a urologist or urogynecologist in an outpatient setting | |
| Requires microbial testing according to clinician judgment | |
| Sample ordered with an ICD-10-CM code associated with UTI | |
| Sample contained enough urine to conduct M-PCR/P-AST, SUC, and | |
| biomarker assays on the same sample | |

Subjects' de-identified urine samples were stored in a biorepository and evaluated at Pathnostics' clinical laboratory. The Western Institutional Review Board deemed this remnant sample study to be exempt under 45 CFR § 46.104 (d) (4), as the information is used in a manner such that the identity of the subject cannot be readily ascertained directly or via identifiers linked to the subjects, the subject is not contacted, and the investigator will not re-identify subjects. Urine samples from any previous IRB-approved clinical trials where the patient specifically opted out from research use of their remnant samples and corresponding de-identified data were excluded.

Specimen Handling: All urine specimens in this study were collected into a sterile cup via the midstream clean-catch method. Specimens were split and transferred into two Vacutainers® (Becton Dickinson, Franklin Lakes, NJ, USA), one yellow-top tube for the P-AST assays and one gray-top tube containing boric acid for the M-PCR, SUC, and biomarker testing. Upon receipt, each urine sample was separated into 1 mL aliquots and placed in microcentrifuge tubes labeled with unique codes that did not contain patient identifiers. Labels were placed securely on each tube and scanned into software for biobanking and future tracking. The only data associated with each biobanked sample were the age and sex of the patient and any associated ICD-10-CM codes.

Aliquots immediately underwent testing via M-PCR and SUC. Aliquots for biomarker (NGAL, IL-8, and IL-1β) testing were centrifuged at 13,200 rpm for 2 min. The aspiration of the supernatant was transferred to a clean tube, labeled, and frozen at −80° C.+/−10° C. until ELISA testing.

Specimen Testing: Enzyme-linked immunosorbent assay (ELISA)-ELISA kits purchased from R&D Systems/Bio-Techne (Minneapolis, MN, USA), including human Lipocalin-2/NGAL Quantikine ELISA Kit (Catalog number SLCN20), human IL-8/CXCL8 Quantikine ELISA Kit (Catalog number S8000C), and human IL-1β/IL-1F2 Quantikine ELISA kit (Catalog number SLB50) were used. The assays measured the levels of NGAL [range 0.2-500 ng/ml], IL-8 [range 7.5-2000 pg/mL], and IL-1β [range 3.9-250 pg/mL] in the urine study specimens per the manufacturer's instructions. An Infinite M Nano+ microplate reader (TECAN, Switzerland) measured absorbance at 450 nm and 540 nm, respectively. Frozen supernatants were thawed at room temperature before assaying.

The multiplex-polymerase chain reaction (M-PCR) and pooled antibiotic susceptibility testing (P-AST), M-PCR/P-AST assays (Guidance® UTI, Pathnostics, Irvine, CA, USA) were used for susceptibility testing for 19 antibiotics, semi-quantitation of 27 pathogens, 3 bacterial groups, the ESBL phenotype, and the identification of 32 antibiotic-resistance genes. This test is intended to be used for the diagnosis of complicated, persistent, or recurrent UTIs Is and for UTIs in elevated-risk patients. First, KingFisher/MagMAX™ automated DNA extraction instrument and the MagMAX™ DNA Multi-Sample Ultra Kit (Thermo Fisher, Carlsbad, CA, USA) extracted microbial DNA from the urine specimen according to the manufacturer's instructions. The extracted DNA was used to identify and quantitate the specimen microbes. After combining a universal PCR master mix and the extracted DNA, amplification was completed using TaqMan® technology in a Life Technologies 12 K Flex 112-format OpenArray System (Thermo Fisher Scientific, Wilmington, NC, USA). The inhibition PCR control used was Bacillus atrophaeus. Plasmids containing bacterial target DNA unique to each microbial species acted as positive controls. Duplicate specimen DNA samples were spotted on 112-format OpenArray chips (Thermo Fisher Scientific, Wilmington, NC, USA). The Pathnostics data analysis tool (Pathnostics, Irvine, CA, USA) sorted data, assessed data quality, summarized control sample data, identified positive assays, quantified bacterial load, and generated results. The results of the antibiotic resistance gene detection and the P-AST component of the test, which provides pooled phenotypic susceptibility results for 19 antibiotics, were not included in this analysis.

Quantitative M-PCR used probes and primers to detect the following microbes (23 bac-teria species, 4 yeast species, and 3 bacterial groups): Acinetobacter baumannii (A. baumannii); Actinotignum schaalii (A. schaalii); Aerococcus urinae (A. urinae); Alloscardovia omnicolens (A. omnicolens); Candida albicans (C. albicans); Candida auris (C. auris); Candida glabrata (C. glabrata); Candida parapsilosis (C. parapsilosis); Citrobacter freundii (C. freundii); Citrobacter koseri (C. koseri); Corynebacterium riegelii (C. riegelil); Enterococcus faecalis (E. faecalis); En-terococcus faecium (E. faecium); Escherichia coli (E. coli); Gardnerella vaginalis (G. vaginalis); Klebsiella oxytoca (K. oxytoca); Klebsiella pneumoniae (K. pneumoniae); Morganella morganii (M. morganii); Mycoplasma hominis (M. hominis); Pantoea agglomerans (P. agglomerans); Proteus mirabilis (P. mirabilis); Providencia stuartii (P. stuartii); Pseudomonas aeruginosa (P. aeruginosa); Serratia marcescens (S. marcescens); Staphylococcus aureus (S. aureus); Streptococcus agalactiae (S. agalactiae); Ureaplasma urealyticum (U. urealyticum); Coagulase Negative Staphylococci (CoNS), which includes Staphylococcus epidermidis, Staphylococcus haemolyticus, Staphylococcus lugdunenesis, and Staphylococcus saprophyticus (S. saprophyticus); the Enterobacter Group, which includes Klebsiella aerogenes (K. aerogenes) (formally known as Enterobacter aerogenes) and Enterobacter cloacae (E. cloacae); and Viridans Group Streptococci (VGS), which includes Streptococcus anginosus, Streptococcus oralis, and Streptococcus pasteuranus. Generated reports provided the name(s) of all yeasts detected at any level and all bacteria detected at a density range of <10,000, 10,000-49,999, 50,000-99,999, or ≥100,000 in cells/mL. The cells/mL quantitation was previously shown to correlate linearly, 1:1, with CFUs/mL as defined by SUC.

A standard urine culture (SUC) was performed. Briefly, urine was vortexed, and samples of 1 μL each were spread onto blood agar and colistin and nalidixic acid agar/MacConkey agar (CNA/MAC) plates, respectively, using a sterile plastic loop. All plates were incubated for 24 h at 35° C. under aerobic conditions. Plates with <10,000 CFUs/mL were reported as normal urogenital flora, and plates with growth≥10,000 CFU/mL were used for colony counts (blood agar plates) and identification and quantitation of each morphologically distinct and separate colony (CNA/MAC plates). If ≥3 pathogens were present without a predominant species, results were reported as contaminated/mixed flora. Pathogen identification was confirmed via the VITEK 2 Compact System (bioMerieux, Durham, NC, USA).

Statistical analysis: Participant demographics and the ICD-10-CM code breakdown are described in the summary statistics table (e.g., mean and standard deviation (SD) for continuous variables, such as age, or count and percentage for categorical variables, such as sex and ICD-10-CM. The distribution of all the microorganisms listed above is provided with the number of each species detected and population percentages via M-PCR and by SUC. Summary statistics (n, median, mean) for the expression of the three biomarkers are provided for different sub-groups: microbial density and detection method, either M-PCR or SUC. Each subgroup median value was compared to the “no microbes detected” group median using the Wilcoxon test. Previously published thresholds for biomarker positivity were used as cutoffs for the analysis:
NGAL≥38 ng/ml, IL-8≥20.6 pg/mL, and IL-1β≥12.4 pg/mL. This study defined biomarker consensus as any two or all three of the biomarkers positive at or above the cutoff levels. Individual biomarker results were evaluated with summary statistics (n, median, mean). Results were compared between different microbial density categories derived via both M-PCR and SUC. All hypothesis tests were two-sided, and a p-value of <0.05 was considered statistically significant. All data analyses were performed using R 4.2.2

Patient Demographics: Urine samples were examined from a total of 583 individuals with a mean age of 76.6 years (standard deviation 8.85, median 76.3, range 60.0-99.7)). Of these, 68.3% (n=398) were females, and 31.7% (n=185) were males. Most symptomatic patient samples were submitted with an ICD-10-CM code of N39.0 for “Urinary tract infection, site not specified” [81.8% (n=534)]; see Table 1.

TABLE 1
Top ICD-10-CM codes from the symptomatic study cohort.

| ICD-10-CM Code | Code Description | Frequency | Percent |
|---|---|---|---|
| N39.0 | Urinary tract infection, site not specified | 534 | 81.8 |
| R30.0 | Dysuria | 43 | 6.6 |
| R31.0 | Gross hematuria | 25 | 3.8 |
| Z87.440 | Personal history of diseases of urinary system | 5 | 0.8 |
| R31.9 | Hematuria, unspecified | 4 | 0.6 |
| R82.998 | Other abnormal findings in urine | 4 | 0.6 |
| Others | | 38 | 12.4 |

Some patients had more than one ICD-10-CM code associated with their case. The five most prevalent codes are listed individually, and all remaining codes are grouped together as “other”.

Bacterial and Yeast Identification: Out of the 583 urine samples obtained from patients with UTI symptoms, M-PCR did not detect any microbes in 117 samples, while SUC did not detect any microbes in 193 samples. In samples in which microorganisms were detected, M-PCR identified 883 microbes with densities of ≥10,000 cells/mL, while SUC identified 496 microorganisms at a threshold of ≥10,000 CFU (Table 3).

Biomarker Detections: Specimen levels of NGAL, IL-8, and IL-1β biomarkers were measured via ELISA. These levels were plotted against four ranges of microbial densities detected via M-PCR and SUC: no microorganisms detected; <10,000, 10,000 to 99,999; and ≥100,000 cells or CFU per mL. Levels of each biomarker versus density ranges are shown in FIG. 1A-1C. The “no microorganisms detected” group was used for statistical comparisons, employing the Wilcoxon test to evaluate the differences between the biomarker level medians from each of the microbe density ranges. Table 4 presents the comprehensive summary of comparisons between each of the three biomarkers and density categories from both PCR and SUC.

In both the M-PCR and SUC methodologies, there was a consistent rise in the median levels of the three biomarkers as the microbial density increased. Compared to the group with no microbes detected, biomarkers showed significantly higher median levels, starting in the 10,000 to 99,999 cells/mL category for M-PCR (p=0.0002, p<0.0001, p<0.0001) and SUC (p=0.0104, p=0.0014, p=0.0064) for NGAL, IL-8, and IL-1β, respectively. The ≥100,000 cells/mL category also displayed a significantly higher median level of each biomarker according to both M-PCR (p<0.0001 for all) and SUC (p<0.0001 for all). There was no significant difference in the median levels between specimens with no microbes detected and specimens with microbes detected at <10,000 cells/mL for M-PCR (p=0.2076, p=0.7018, p=0.86) or CFU/mL for SUC (p=0.8056, p=0.7767, p=0.2083) for NGAL, IL-8, or IL-1β, respectively.

TABLE 4
Summary Descriptive Statistics of NGAL, IL-8, and IL-1β Biomarker
Levels Stratified by Microbial Density as Detected by M-PCR and SUC.

| Microbial | M PCR | | | | SUC | | | |
|---|---|---|---|---|---|---|---|---|
| Density Range | n | Median | Mean | p-value | n | Median | Mean | p-value |

NGAL

| | | | | | | | | |
|---|---|---|---|---|---|---|---|---|
| No Microbes | 117 (20.0%) | 16.05 | 56.68 | — | 193 (33.1%) | 33.49 | 120.71 | — |
| Detected | | | | | | | | |
| <10,000 | 23 (3.9%) | 34.55 | 105.03 | 0.2076 | 12 (2.1%) | 39.44 | 100.12 | 0.8056 |
| 10,000-99,999 | 79 (13.6%) | 53.78 | 145.3 | 0.0002 | 115 (19.7%) | 65.49 | 152.44 | 0.0104 |
| ≥100,000 | 364 (62.4%) | 228.87 | 260.27 | <0.0001 | 263 (45.1%) | 268.81 | 278.46 | <0.0001 |

IL-8

| | | | | | | | | |
|---|---|---|---|---|---|---|---|---|
| No Microbes | 117 (20.0%) | 23.95 | 236.57 | — | 193 (33.1%) | 73.84 | 354.64 | — |
| Detected | | | | | | | | |
| <10,000 | 23 (3.9%) | 31.22 | 312.06 | 0.7018 | 12 (2.1%) | 63.30 | 253.22 | 0.7757 |
| 10,000-99,999 | 79 (13.6%) | 141.84 | 531.96 | <0.0001 | 115 (19.7) | 164.57 | 523.82 | 0.0014 |
| ≥100,000 | 364 (62.4%) | 37.12 | 685.69 | <0.0001 | 263 (45.1%) | 431.6 | 740.5 | <0.0001 |

IL-1β

| | | | | | | | | |
|---|---|---|---|---|---|---|---|---|
| No Microbes | 117 (20.0%) | 3.9 | 18357 | — | 193 (33.1%) | 3.9 | 38.17 | — |
| Detected | | | | | | | | |
| <10,000 | 23 (3.9%) | 3.9 | 13.86 | 0.86 | 12 (2.1%) | 3.9 | 22.09 | 0.2083 |
| 10,000-99,999 | 79 (13.6%) | 4.08 | 57.95 | <0.0001 | 115 (19.7) | 11.38 | 62.75 | 0.0064 |
| ≥100,000 | 364 (62.4%) | 47.85 | 93.4 | <0.0001 | 263 (45.1%) | 57.88 | 99.69 | <0.0001 |

*p-values are relative to symptomatic no bacteria detected
*p-values calculated based on the medians

Biomarker Patterns: Microbial Density and Technique Differences: As observed in Table 4 and FIG. 1A-1C, a consistent correlation exists between increased microbial density and elevated biomarker levels, regardless of the microbial detection method used. There was a significantly higher number of SUC specimens with negative results (n=193) compared to M-PCR specimens (n=117).

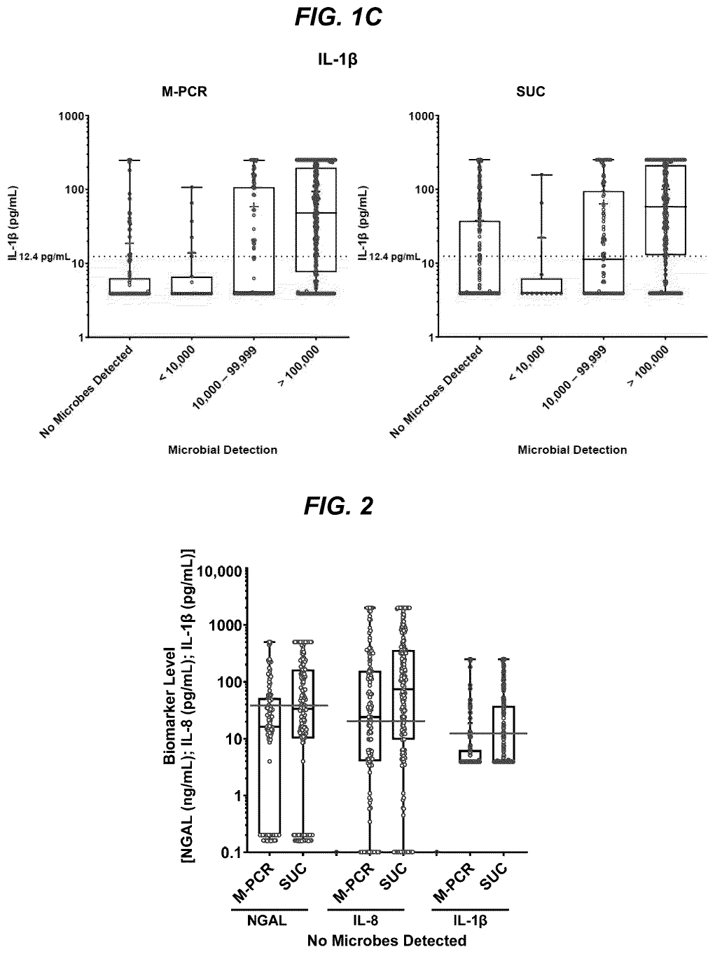

Median biomarker levels in the “no microorganisms detected” group were significantly higher for NGAL (p=0.002), IL-8 (p=0.009), and IL-1β (p=0.001) when microbial detection was assessed via SUC compared to M-PCR. (FIG. 2). There was also a higher number of specimens identified with microbial densities in the ≥100,000 cells/mL category when tested via M-PCR (n=364) compared to SUC (n=263). Several specimens from the “no microorganisms detected” SUC group had high biomarker levels and high microbial densities when tested via M-PCR. Out of the 193 samples that were negative based on SUC, 60 (31%) showed high levels of both microbes, ≥10,000 from M-PCR and NGAL (above threshold); 70 (36.2%) had elevated microbial levels, >10,000 from M-PCR, along with high levels of IL-8 (above threshold); and 47 (44.4%) had microbial levels>10,000 by M-PCR combined with biomarker levels that surpassed the threshold for IL-1β (above threshold).

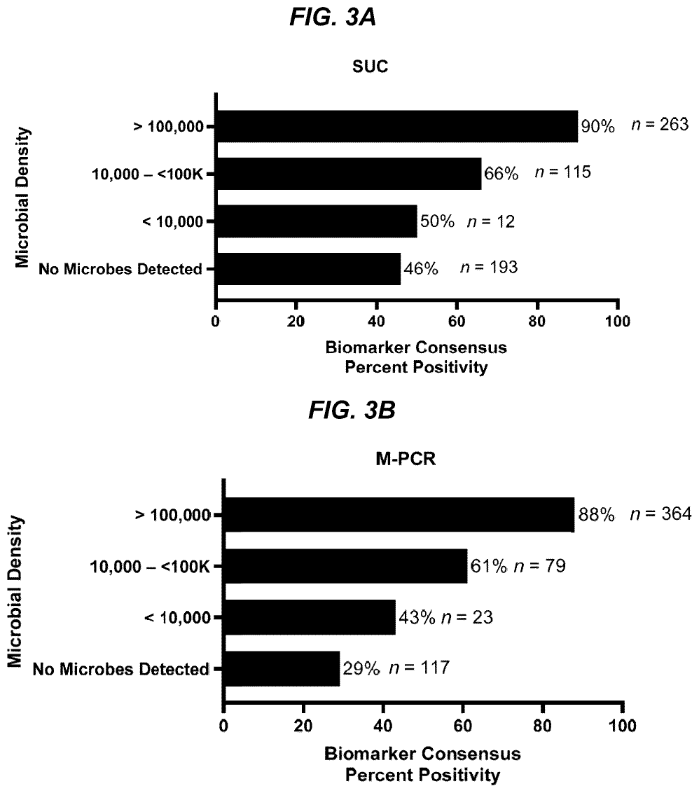

Biomarker Consensus by Microbial Density: The assessment of each specimen for biomarker consensus (defined as two or more biomarkers above the established cutoffs) was conducted and organized based on microbial density and detection method (FIGS. 3A and 3B). Specimens having microbial densities≥10,000 cells/mL detected via either M-PCR or SUC exhibited over 50% biomarker consensus positivity with the percent of positivity increasing as microbial density rose. SUC-negative cases, in which no microbes were detected (n=193), had biomarker consensus-positive results in 46% of cases, which was much higher than M-PCR-negative cases (n=117) with 29% being consensus-positive.

The current standard-of-care SUC method and the culture-independent M-PCR assay was employed to identify and quantify microbes from patients with presumptive UTIs. Previous studies have demonstrated a 1:1 correlation for microbial quantitation in the linear range between SUC and M-PCR, allowing for direct comparisons between culture-dependent and culture-independent methods across various microbial densities. Additionally, the immune response was assessed by measuring infection-associated biomarkers (NGAL, IL-8, and IL-1β) within the same urine specimens. This unique approach facilitated meaningful comparisons between SUC and M-PCR and provided direct associations between the presence and density of microorganisms against the immune response in clinically relevant specimens.

Traditionally, a microbial density of ≥100,000 cells/mL has been deemed diagnostically significant; however, recent clinical reviews and guidelines have proposed lower thresholds that are still clinically relevant. Lower microbial densities (>10,000 cells/mL) detected via M-PCR and SUC in symptomatic subjects showed a notable increase in infection-associated biomarker levels. Additionally, for these subjects suspected of having a UTI, a cell density of ≥10,000 cells/mL from M-PCR and SUC strongly correlated with biomarker consensus. These findings suggest that a microbial detection threshold of 10,000 cells/mL could be an indicative criterion for diagnosing a UTI. Using a threshold of >100,000 cells/mL as a criterion for initiating antimicrobial therapy carries significant clinical implications. One consequence is the potential for undertreatment of certain UTI patients, allowing microbes to proliferate, infiltrate host cells, develop biofilms, or acquire antibiotic resistance before treatment is administered. Delaying treatment can lead to a progression of clinical severity, potentially necessitating higher antibiotic doses for extended durations or increasing the likelihood of complications, including recurrent infections and bacteremia.

Building on these findings, more specimens in which no microorganisms were detected (n=193) in the SUC group compared to the M-PCR group (n=117). Interestingly, the specimens with no microorganisms detected via SUC exhibited significantly higher levels of NGAL, IL-8, and IL-1β than those with no microbes detected via M-PCR. Furthermore, SUC specimens in which no organisms were detected demonstrated significantly higher (46%) consensus scores (two or more biomarkers above the established thresholds) when compared to M-PCR negative cases (29%).

The significantly higher biomarker levels in SUC-negative cases compared to M-PCR-negative cases have important implications. Firstly, the difference in biomarker levels underscores the potential variation in effectiveness between SUC and M-PCR as testing methods for detecting potential pathogens, suggesting that M-PCR may have higher sensitivity and specificity for detecting microbes that are causing a UTI, thereby enhancing diagnostic accuracy for symptomatic cases of UTI. Furthermore, SUC cases displaying low or no microbial densities alongside high biomarker levels suggest a potential failure in detecting the organisms causing the UTI, leading to these elevated levels. These findings challenge the sensitivity of SUC for the identification of uropathogens, raising questions about false negatives in culture-based testing. Consequently, the increase in biomarker levels between SUC-negative cases highlights the need to carefully consider the testing method employed when interpreting results and making treatment decisions, as SUC-negative cases may warrant closer monitoring.

In conclusion, this example shows that symptomatic subjects with UTIs exhibit a signifi-cant immune response at a microbial density threshold of ≥10,000 cells/mL, regardless of the detection method used. This suggests that a lower diagnostic microbial density threshold is clinically appropriate for UTI diagnosis and management, applicable to both the microbial identification and quantitation techniques used here.

### Example 2

The following is a non-limiting example of the present invention. It is to be understood that said example is not intended to limit the present invention in any way. Equivalents or substitutes are within the scope of the present invention.

The current UTI diagnostic, standard urine culture (SUC), favors the cultivation of easy-to-grow Gram-negative microbes such as Escherichia coli. However, this method is less favorable for the growth of non-E. coli microorganisms and is completely inadequate for the growth of fastidious microbes, which are increasingly being recognized as common uropathogens. Additionally, the SUC method is significantly less likely to identify polymicrobial infections.

Example 2 correlates the detection of microbes by M-PCR and SUC with cases with elevated levels of infection-associated urine biomarkers in individuals diagnosed with UTIs in a urology setting. The goal was to determine whether M-PCR is over-reporting microbes in cases that are not UTIs or whether SUC is under-reporting microbes in cases that are clearly UTIs.

Study Design: This study compared biomarker levels in symptomatic patients with and without microorganisms detected in their urine by SUC and M-PCR. The purpose was to determine whether organisms detected by M-PCR represent active UTIs as defined by elevated biomarker levels in the urine of these presumptive UTI cases, addressing the concern that M-PCR testing may result in overdiagnosis.

The cohort consisted of 583 individuals aged 60 yr and older who presented at urology clinics in 39 US states. All participants were assigned ICD-10-CM codes in the urology specialty setting based on the clinical presentation of the patient. Only specimens sent for UTI diagnostic testing with ICD-10-CM codes for either UTIs or UTI-related conditions were selected for this study. Specimens were included from consecutive eligible patients and collected between Jan. 17, 2023, and Apr. 24, 2023.

Each de-identified urine sample was assigned a repository label associated with a record of the participant's age, sex, and ICD-10-CM code(s), and stored in a biorepository for evaluation at Pathnostics' (Irvine, CA, USA) clinical laboratory. The Western Institutional Review Board deemed this remnant sample study to be exempt under 45 CFR § 46.104 (d) (4) as the information was used in a manner that the identity of the participant could not be readily ascertained directly or through identifiers linked to the participants, the participant was not contacted, and the investigator did not reidentify the participants. Urine samples from any previous institutional review board-approved clinical trials where the patient specifically opted out from research use of their remnant samples and corresponding de-identified data were excluded. Upon receipt at the testing laboratory (Pathnostics), each urine specimen was processed for microbial identification by M-PCR and SUC, and for a biomarker analysis by enzyme-linked immunosorbent assay (ELISA).

Specimen testing closely resembles the methodology outlined in Example 1.

Participant demographics and ICD-10-CM codes: The cohort consisted of 583 unique patients, predominantly female (68.3%, n=398), whose ages ranged from 60.0 to 99.7 yr, with a mean of 76.6 yr (standard deviation=8.87) and a median of 76.3 yr of age (Table 5). The most prevalent ICD-10-CM code was N39.0 for “UTI, site not specified” (n=534, 81.8%; Table 6).

TABLE 5
Demographics of the Study Cohort.
Total Subjects (n = 583)

| Age | | Sex | |
|---|---|---|---|
| Mean (SD) | 76.6 (8.85) | Female | 398 (68.3%) |
| Median [Min, Max] | 76.3 [60.0, 99.7] | Male | 185 (31.7%) |

TABLE 6
Summary of Most Prevalent ICD-10-CM
Codes for the Study Cohort

| Description | Monomicrobial | Polymicrobial | Overall |
|---|---|---|---|
| Urinary tract infection, | 172 (83.1%) | 260 (87.0%) | 534 (81.8%) |
| site not specified | | | |
| Dysuria | 10 (4.8%) | 13 (4.3%) | 43 (6.6%) |
| Gross hematuria | 9 (4.3%) | 4 (1.3%) | 25 (3.8%) |
| — | 15 (7.5%) | 20 (6.7%) | 50 (7.6%) |

Bacterial and yeast identification by M-PCR and SUC: M-PCR identified 883 microorganisms in the 583 specimens, indicating that many specimens (40%, n=231) contained two or more microorganisms (polymicrobial; Table 3 Example 1). E. coli was detected in 188 specimens (32%) and non-E. coli microorganisms were detected in 221 specimens (38%). SUC identified 496 microorganisms in the 583 specimens (Supplementary Table 3). It detected E. coli in 160 specimens (27%) and non-E. coli microorganisms in 171 specimens (29%).

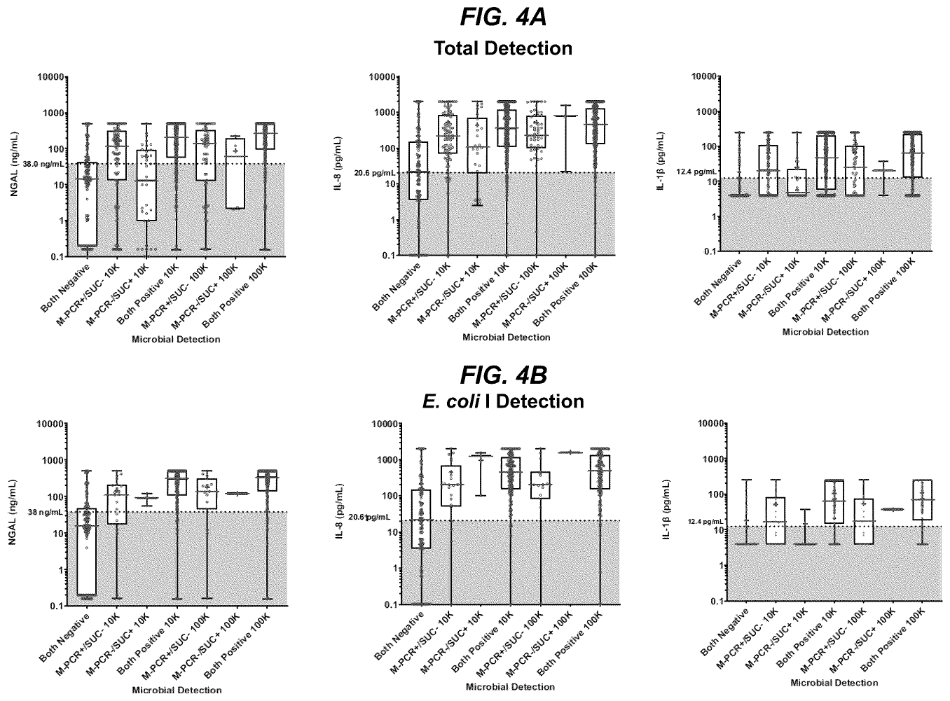

Biomarker levels correlate with microbial detection: Results of microbial detection were categorized into four groups: M-PCR-positive/SUC-negative (n=86), M-PCR-negative/SUC-positive (n=26), M-PCR-positive/SUC-positive (n=351), and M-PCR-negative/SUC-negative (n=120). Specimens with M-PCR negative/SUC negative results were considered negative for UTIs. NGAL, IL-8, and IL-1β biomarker levels in specimens with microbial densities of ≥10 000 cells/ml and ≥100 000 cells/ml were compared with microbe-negative specimens (FIG. 4A). Biomarker results were further stratified into groups by microbes identified (E. coli, non-E. coli, and polymicrobial) to evaluate their impact on biomarker levels (FIG. 4B-4D, respectively).

M-PCR-positive/SUC-positive cases at microbial density thresholds of ≥10 000 cells/ml (n=351) or ≥100 000 cells/ml (n=244) had significantly elevated levels of all three biomarkers compared with M-PCR-negative/SUC-negative cases (p<0.0001; FIG. 4A and Table 7). At both ≥10 000 cells/ml and ≥100 000 cells/ml, M-PCR-positive/SUC-negative cases (n=86 and n=52, respectively) also had significantly elevated levels of all three biomarkers (p≤0.005). However, biomarker levels in M-PCR-negative/SUC-positive cases (n=26) at a microbial density of ≥10 000 cells/ml were elevated significantly for IL-8 (p=0.006) and IL-1β (p=0.021), but not for NGAL (p=0.15).

| | 10K cells/mL or CFUs/mL | | | 100K cells/mL or CFUs/mL | | |
|---|---|---|---|---|---|---|
| | SUC−/ | SUC+/ | Both | SUC−/ | SUC+/ | Both |
| Negative | MPCR+ | MPCR− | Positive | MPCR+ | MPCR− | Positive |

NGAL (ng/mL)

| | | | | | | | |
|---|---|---|---|---|---|---|---|
| n | 120 | 86 | 26 | 351 | 52 | 2 | 244 |
| 25th | 0.2 | 38.77 | 9.8 | 64.64 | 50.24 | — | 104.72 |
| percentile | | | | | | | |
| Median | 15.84 | 147.02 | 51.72 | 211.09 | 161.22 | — | 283.24 |
| 75th | 40.12 | 327.47 | 114.3 | 500.00 | 379.46 | — | 500.0 |
| percentile | | | | | | | |
| Lower 95% | 42.62 | 157.46 | 37.9 | 231.47 | 165.78 | — | 261.88 |
| CI of mean | | | | | | | |
| Mean | 65.09 | 196.63 | 82.79 | 251.83 | 216.78 | — | 285.5 |
| Upper 95% | 87.55 | 236.1 | 127.7 | 272.19 | 268.12 | 309.13 | 301.36 |
| CI of mean | | | | | | | |
| p-values vs | — | <0.0001 | 0.15 | <0.0001 | <0.0001 | — | <0.0001 |
| negative | | | | | | | |

IL-8 (pg/mL)

| | | | | | | | |
|---|---|---|---|---|---|---|---|
| n | 120 | 86 | 26 | 351 | 52 | 2 | 244 |
| 25th | 3.62 | 71.36 | 24.77 | 109.12 | 103.15 | — | 103.11 |
| percentile | | | | | | | |
| Median | 21.47 | 219.54 | 108.1 | 355.32 | 228.5 | — | 450.23 |
| 75th | 147.14 | 818.19 | 603.2 | 1206.4 | 815.02 | — | 1306.5 |
| percentile | | | | | | | |
| Lower 95% | 126.14 | 393.38 | 193.5 | 618.54 | 357.38 | — | 653.68 |
| CI of mean | | | | | | | |
| mean | 213.89 | 533.17 | 444.1 | 693.47 | 532.88 | — | 744.02 |
| Upper 95% | 301.36 | 672.96 | 694.8 | 76.4 | 708.38 | — | 834.36 |
| CI of mean | | | | | | | |
| p-values vs | — | <0.0001 | 0.006 | <0.0001 | <0.0001 | — | <0.0001 |
| negative | | | | | | | |

IL-1β (pg/mL)

| | | | | | | | |
|---|---|---|---|---|---|---|---|
| n | 120 | 86 | 26 | 351 | 52 | 2 | 244 |
| 25th | 3.9 | 3.9 | 3.9 | 5.67 | 3.9 | — | 12.93 |
| percentile | | | | | | | |
| Median | 3.9 | 20.71 | 4.72 | 47.07 | 25.37 | — | 63.29 |
| 75th | 3.975 | 106.21 | 20.13 | 204.32 | 94.08 | — | 217.14 |
| percentile | | | | | | | |
| Lower 95% | 10.3 | 46.06 | 3.65 | 83.14 | 42.47 | — | 89.73 |
| CI of mean | | | | | | | |
| mean | 18.45 | 63.05 | 24.85 | 93.4 | 65.32 | — | 102.07 |
| Upper 95% | 26.6 | 80.04 | 46.05 | 103.67 | 88.17 | — | 114.41 |
| CI of mean | | | | | | | |
| p-values vs | — | <0.0001 | 0.021 | <0.0001 | <0.0001 | — | <0.0001 |
| negative | | | | | | | |

The Mann-Whitney test was used to compare biomarker levels from each detection method/microbial density group to the microbe-negative group. A p-value<0.05 was considered statistically significant.

E. coli detection: Cases in which E. coli was detected by both SUC and M-PCR at ≥10 000 cells/ml (n=157) or ≥100 000 cells/ml (n=122; FIG. 4B and Supplementary Table 5) had significantly elevated levels of all three biomarkers (p<0.0001), as well as cases in which M-PCR was positive and SUC was negative (n=21 and n=15, at 10 000 and 100 000 density thresholds, respectively, p≤0.005). Only three cases occurred in which SUC was positive for E. coli and M-PCR was negative, which had statistical significance only for the elevation of IL-8 (p=0.035), and only one was observed when using the 100 000 cells/ml microbial density threshold.

TABLE 8
Descriptive Statistics for Biomarker Levels with Microbial
Detection of E. coli Cases by SUC and M-PCR

| | 10K cells/mL or CFUs/mL | | | 100K cells/mL or CFUs/mL | | |
|---|---|---|---|---|---|---|
| | SUC−/ | SUC+/ | Both | SUC−/ | SUC+/ | Both |
| Negative | MPCR+ | MPCR− | Positive | MPCR+ | MPCR− | Positive |

NGAL (ng/mL)

| | | | | | | | |
|---|---|---|---|---|---|---|---|
| n | 120 | 21 | 3 | 157 | 15 | 1 | 122 |
| 25th | 0.2 | 21.89 | 74.04 | 104.77 | 58.0 | — | 144.22 |
| percentile | | | | | | | |
| Median | 15.84 | 112.44 | 91.85 | 308.0 | 136.98 | — | 342.1 |
| 75th | 40.12 | 202.6 | 106.2 | 500.0 | 261.27 | — | 500.0 |
| percentile | | | | | | | |
| Lower 95% | 42.62 | 78.46 | 9.47 | 261.56 | 94.81 | — | 280.75 |
| CI of mean | | | | | | | |
| Mean | 65.09 | 145.12 | 89.55 | 291.01 | 181.04 | — | 313.44 |
| Upper 95% | 87.55 | 211.96 | 169.64 | 320.47 | 267.28 | — | 346.12 |
| CI of mean | | | | | | | |
| p-values vs | — | 0.005 | 0.062 | <0.0001 | 0.001 | — | <0.0001 |
| negative | | | | | | | |

IL-8 (pg/mL)

| | | | | | | | |
|---|---|---|---|---|---|---|---|
| n | 120 | 21 | 3 | 157 | 15 | 1 | 122 |
| 25th | 3.62 | 55.53 | 675.6 | 153.69 | 93.16 | — | 156.71 |
| percentile | | | | | | | |
| Median | 21.47 | 201.35 | 1251 | 446.49 | 201.35 | — | 484.92 |
| 75th | 147.14 | 456.39 | 1402 | 1130.2 | 410.79 | — | 1308.0 |
| percentile | | | | | | | |
| Lower 95% | 126.14 | 179.26 | 936.2 | 605.72 | 109.29 | — | 630.63 |
| CI of mean | | | | | | | |
| mean | 213.89 | 443.38 | 968.5 | 714.2 | 442.29 | — | 757.7 |
| Upper 95% | 301.36 | 707.51 | 3837.1 | 822.69 | 775.26 | — | 884.77 |
| CI of mean | | | | | | | |
| p-values vs | — | <0.0001 | 0.035 | <0.0001 | 0.002 | — | <0.0001 |
| negative | | | | | | | |

IL-1β (pg/mL)

| | | | | | | | |
|---|---|---|---|---|---|---|---|
| n | 120 | 21 | 3 | 157 | 15 | 1 | 122 |
| 25th | 3.9 | 3.9 | 3.9 | 15.1 | 5.44 | — | 20.09 |
| percentile | | | | | | | |
| Median | 3.9 | 17.03 | 3.9 | 63.04 | 17.25 | — | 71.38 |
| 75th | 3.975 | 76.94 | 20.45 | 235.9 | 73.2 | — | 246.48 |
| percentile | | | | | | | |
| Lower 95% | 10.3 | 19.57 | 32.54 | 90.41 | 11.84 | — | 92.12 |
| CI of mean | | | | | | | |
| mean | 18.45 | 52.14 | 14.49 | 106.13 | 53.88 | — | 109.87 |
| Upper 95% | 26.6 | 84.7 | 62.14 | 121.85 | 95.91 | — | 127.61 |
| CI of mean | | | | | | | |
| p-values vs | — | <0.0001 | 0.723 | <0.0001 | <0.0001 | — | <0.0001 |
| negative | | | | | | | |

Detection of non-E. coli microbes: Cases in which non-E. coli microorganisms were detected by both SUC and M-PCR at ≥10 000 cells/ml or CFUs/ml (n=172) or ≥100 000 cells/ml (n=104; FIG. 4C and Table 9) had significantly elevated levels of all three biomarkers (p<0.0001). Cases in which M-PCR was positive, and SUC was negative (n=65 and n=37, at ≥10 000 and ≥100 000 cells/ml, respectively) also had significantly elevated levels of all three biomarkers (p<0.0001). However, biomarker levels in M-PCR-negative/SUC-positive cases (n=23) at a microbial density of ≥10 000 cells/ml were significantly elevated for IL-8 (p=0.026) and IL-1β (p=0.018) only, but not for NGAL (p=0.366).

TABLE 9
Descriptive Statistics for Biomarker Levels with Detection
of Non-E. coli Microorganisms by SUC and M-PCR

| | 10K cells/mL or CFUs/mL | | | 100K cells/mL or CFUs/mL | | |
|---|---|---|---|---|---|---|
| | SUC−/ | SUC+/ | Both | SUC−/ | SUC+/ | Both |
| Negative | MPCR+ | MPCR− | Positive | MPCR+ | MPCR− | Positive |

NGAL (ng/mL)

| | | | | | | | |
|---|---|---|---|---|---|---|---|
| n | 120 | 65 | 23 | 172 | 37 | 1 | 104 |
| 25th | 0.2 | 49.12 | 4.97 | 41.65 | 50.62 | — | 95.21 |
| percentile | | | | | | | |
| Median | 15.84 | 158.57 | 37.15 | 140.8 | 162.85 | — | 204.56 |
| 75th | 47.62 | 481.9 | 112.2 | 495.1 | 200.0 | — | 500.0 |
| percentile | | | | | | | |
| Lower 95% | 37.0 | 165.49 | 30.86 | 190.8 | 166.28 | — | 225.12 |
| CI of mean | | | | | | | |
| Mean | 65.09 | 213.25 | 81.91 | 231.27 | 231.27 | — | 262.35 |
| Upper 95% | 87.55 | 261.0 | 133.0 | 296.26 | 296.26 | — | 299.57 |
| CI of mean | | | | | | | |
| p-values vs | — | <0.0001 | 0.366 | <0.0001 | 0.001 | — | <0.0001 |
| negative | | | | | | | |

IL-8 (pg/mL)

| | | | | | | | |
|---|---|---|---|---|---|---|---|
| n | 120 | 65 | 23 | 172 | 37 | 1 | 104 |
| 25th | 3.62 | 73.84 | 16.22 | 80.22 | 105.83 | — | 136.76 |
| percentile | | | | | | | |
| Median | 21.47 | 252.88 | 93.67 | 317.08 | 316.85 | — | 485.15 |
| 75th | 147.14 | 818.39 | 400.4 | 1257.1 | 817.59 | — | 1342.8 |
| percentile | | | | | | | |
| Lower 95% | 126.14 | 394.87 | 122.9 | 588.67 | 354.17 | — | 626.21 |
| CI of mean | | | | | | | |
| mean | 213.89 | 562.17 | 375.8 | 700.3 | 569.62 | — | 767.86 |
| Upper 95% | 301.36 | 729.48 | 628.6 | 811.94 | 785.07 | — | 909.51 |
| CI of mean | | | | | | | |
| p-values vs | — | <0.0001 | 0.026 | <0.0001 | <0.0001 | — | <0.0001 |
| negative | | | | | | | |

IL-1β (pg/mL)

| | | | | | | | |
|---|---|---|---|---|---|---|---|
| n | 120 | 65 | 23 | 172 | 37 | 1 | 104 |
| 25th | 3.9 | 3.9 | 3.9 | 3.9 | 3.9 | — | 12.0 |
| percentile | | | | | | | |
| Median | 3.9 | 27.36 | 5.54 | 34.75 | 31.93 | — | 64.42 |
| 75th | 3.975 | 107.51 | 17.85 | 161.66 | 107.2 | — | 204.44 |
| percentile | | | | | | | |
| Lower 95% | 10.3 | 46.31 | 2.14 | 70.88 | 41.62 | — | 81.01 |
| CI of mean | | | | | | | |
| mean | 18.45 | 66.58 | 26.14 | 85.25 | 69.96 | — | 99.8 |
| Upper 95% | 26.6 | 86.84 | 50.15 | 99.62 | 98.3 | — | 118.58 |
| CI of mean | | | | | | | |
| p-values vs | — | <0.0001 | 0.018 | <0.0001 | <0.0001 | — | <0.0001 |
| negative | | | | | | | |

The Mann-Whitney test was used to compare biomarker levels from each detection method/microbial density group compared to the microbe-negative group. A p-value<0.05 was considered statistically significant.

Detection of polymicrobial cases: Polymicrobial cases, those in which M-PCR detected two or more microorganisms at >10 000 cells/ml (n=231), also had significantly elevated levels of all three biomarkers (p<0.0001) compared with cases in which no microorganisms were detected by either SUC or M-PCR (n=120; FIG. 4D and Table 10).

TABLE 10
Descriptive Statistics for Biomarker Levels with Polymicrobial Detection by M-PCR

| | NGAL (ng/mL) | | IL-8 (pg/mL) | | IL-1β (pg/mL) | |
|---|---|---|---|---|---|---|
| | M-PCR | | M-PCR | | M-PCR | |
| | Negative | Polymicrobial | Negative | Polymicrobial | Negative | Polymicrobial |
| n | 146 | 231 | 146 | 231 | 146 | 231 |
| 25th | 0.2 | 65.83 | 4.08 | 98.61 | 3.9 | 3.9 |
| percentile | | | | | | |
| Median | 16.37 | 176.51 | 30.23 | 296.1 | 3.9 | 37.95 |
| 75th | 57.82 | 500.0 | 159.84 | 965.16 | 6.66 | 143.03 |
| percentile | | | | | | |
| Lower 95% | 48.31 | 216.02 | 170.46 | 519.24 | 12.0 | 69.63 |
| CI of mean | | | | | | |
| Mean | 68.24 | 241.01 | 254.89 | 606.93 | 19.59 | 81.94 |
| Upper 95% | 88.17 | 266.0 | 339.32 | 694.61 | 27.17 | 93.94 |
| CI of mean | | | | | | |
| p-values vs | — | <0.0001 | — | <0.0001 | — | <0.0001 |
| negative | | | | | | |

The Mann-Whitney test was used to compare biomarker levels from each detection method/microbial density group to the microbe-negative group. A p-value<0.05 was considered statistically significant.

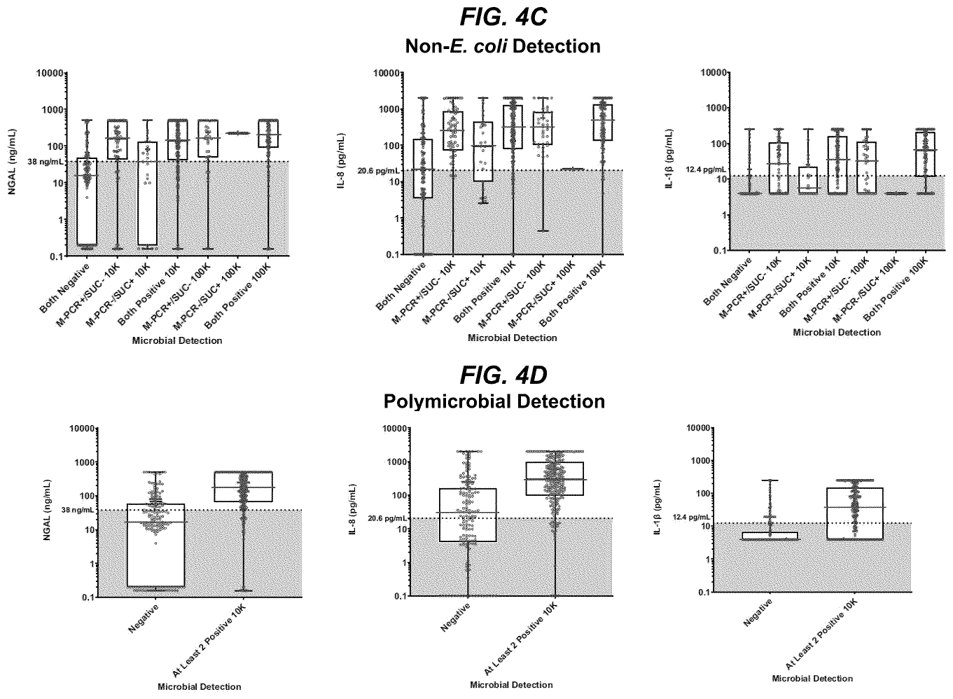

M-PCR detects more biomarker-positive UTIs than SUC: The three infection-associated biomarkers, IL-8 had the highest sensitivity (91.2%) and IL-1β had the highest specificity (96.9%) for UTIs. Biomarker consensus, in which two or more biomarkers were positive, provided an ideal balance of sensitivity (84.0%) and specificity (91.2%). Biomarker percent positivity rates were examined between M-PCR-positive/SUC-negative and M-PCR-negative/SUC-positive cases at a microbial density threshold of >10 000 cells/ml in the symptomatic patient study group of 583 individuals, and stratified biomarker results by the presence or absence of detectable E. coli and by the presence of polymicrobial infection, regardless of microbial species (FIG. 5).

Of all 86 M-PCR-positive/SUC-negative specimens, 76% overall and 77% with non-E. coli microorganisms had two or more positive biomarkers. In contrast, for all 23 M-PC R-negative/SUC-positive specimens, 62% overall and 57% with non-E. coli microorganisms had two or more positive biomarkers (FIG. 5). For cases in which E. coli was detected, 71% (15/21) of M-PCR-positive/SUC-negative specimens had two or more positive biomarkers. Although there were only three M-PCR-negative/SUC-positive specimens with E. coli identified, these all met the criteria for consensus biomarker positivity (FIG. 5).

The development of accurate and rapid diagnostic testing presents an opportunity to improve antibiotic stewardship and reduce healthcare costs by optimizing directed treatment and reducing empiric antibiotic use. Previous retrospective studies have demonstrated that approximately $64,239 in health care expenditures is averted when a single patient avoids hospitalization and/or emergency department visits for a UTI and that the use of M-PCR/P-AST testing to guide management of UTIs was associated with a 13.7% decrease in hospital admissions and/or emergency department utilization when compared with the use of SUC testing (p=0.003). Additionally, among Medicare enrollees, the average total 1-yr UTI-related cost was reportedly $501.85 (95% confidence interval: $79.87, $562.08; p=0.004) lower per patient managed using M-PCR/P-AST versus SUC ($629.55 vs $1131.39), due to lower utilization of hospital, emergency department care, and urgent care.

To answer the question of whether M-PCR testing results in a large number of false positives or whether SUC was underdiagnosing a significant number of UTIs, M-PCR and SUC results were evaluated with levels of three infection-associated biomarkers (NGAL, IL-8, and IL-1β) in the urine of symptomatic patients with a presumptive diagnosis of a UTI from a urology/urogynecology specialty setting. The biomarker levels were compared among specimens with no microorganisms detected by either M-PCR or SUC, with specimens that were M-PCR-positive/SUC-negative, M-PCR-negative/SUC-positive, and M-PCR-positive/SUC-positive. Two thresholds of microbe positivity were evaluated: 100 000 cells/ml or CFUs/ml, which is traditionally considered diagnostically significant for UTIs in the USA, and 10,000 cells/ml or CFUs/ml (see Example 1). Cases where E. coli was detected, non-E. coli organisms were detected, and two or more microorganisms were detected in the same specimen (polymicrobial) were further evaluated.

From the 583 samples tested, 86 were found to be M-PCR-positive/SUC-negative. The median biomarker levels were significantly different for all three biomarkers in this group compared with negative (no infectious organisms detected). Only 26 samples were M-PCR-negative/SUC-positive, and biomarker results were not statistically different from negative, although these were somewhat higher than those of cases that were negative by both tests. Interestingly, the M-PCR-negative/SUC-positive scenario almost exclusively occurred when a threshold of ≥10 000 cells/ml by M-PCR or CFUs/ml by SUC was used and may have been reported as “negative” by SUC according to the current standard practices for the USA, which typically use a threshold of ≥100 000 CFUs/ml.

Across all M-PCR-positive/SUC-negative specimens and in those with non-E. coli organisms, the median level of all three biomarkers (NGAL, IL-8, and IL-1β) was significantly higher (p<0.0001) than in cases in which both M-PCR and SUC were negative, with >75% of positive cases achieving biomarker consensus positivity. M-PCR-positive/SUC-negative specimens with E. coli identified also exhibited elevated median biomarker levels (p≤0.005) compared with dual-negative specimens. M-PCR-negative/SUC-positive specimens had median levels of IL-8 and IL-1b significantly elevated (p<0.05), but these did not exhibit elevated median NGAL levels (p>0.05). This is a strong indication that in cases of disagreement, M-PCR is a more reliable indicator of infection, as indicated by universally elevated median biomarker levels and high biomarker per-cent positivity.

Historically, E. coli has been considered the primary cause of UTIs and was the most frequently detected microbial species by both SUC (n=160) and M-PCR (n=188). However, SUC, which is optimized for the detection of nonfastidious Gram-negative uropathogens such as E. coli, still missed many E. coli cases (12%, n=21), which were detected by M-PCR and had elevated biomarker levels. SUC also failed to detect a significant number of non-E. coli organisms routinely identified by M-PCR. Fastidious organisms, including Aerococcus urinae and Actinotignum schaalii, are being increasingly recognized as uropathogens that may cause or complicate UTIs, especially in high-risk, hospitalized, or elderly patients. Both A. urinae and A. schaalii were among the top five most prevalent organisms identified by M-PCR (n=116 and n=118, respectively; Table 3 in Example 1). Failure to identify these organisms can result in many UTIs going untreated based on negative culture results, potentially prolonging symptoms in patients and resulting in complications, such as urosepsis, in high-risk patients.

In addition, polymicrobial infections are typically either misidentified as monomicrobial, when a single organism dominates SUC, or dismissed as “contaminated samples,” when multiple organisms grow in SUC. Of the 583 specimens in this study, 40% (n=231) had polymicrobial infections with two or more organisms detected at >10 000 cells/ml by M-PCR, and the median biomarker levels in the polymicrobial specimens were significantly elevated (p<0.0001), with 77% achieving biomarker consensus positivity. This indicates that many patients symptomatic for UTIs and tested using SUC may have a polymicrobial infection that is either dismissed as contamination or misdiagnosed as a monomicrobial infection, potentially resulting in suboptimal treatment.

The unique strength of this study was the direct comparison of microbial identity and density results of the same urine specimen using both the current standard of care, SUC, and a novel molecular method, M-PCR, at two microbial density thresholds (10,000 and 100,000 cells/ml or CFUs/ml) combined with the measures of the immune response according to the biomarkers NGAL, IL-8, and IL-1b. This approach allowed us to directly associate the presence and density of microorganisms with infection-associated immune responses in the urinary tract of each patient to make comparisons between detection methods. The use of a large study population recruited through urology offices across 39 states in the continental USA further strengthened the study.

The evidence provided by this study that microorganisms detected by M-PCR correlate with the biomarkers of infection counter the concern that M-PCR overdiagnoses UTIs. Significant debate exists regarding the validity of M-PCR-positive/SUC-negative case results. The findings of this study indicate that >75% of M-PCR-positive/SUC-negative cases are true UTIs, as evidenced by elevated levels of urinary biomarkers in a symptomatic population with a presumptive UTI diagnosis from a urology setting. Many of these infections either were caused by organisms other than E. coli, especially fastidious organisms, or were polymicrobial in nature. The low sensitivity of SUC for detecting these cases, combined with a slower time to results, makes it important to strongly consider advanced UTI tests that provide improved results. This study indicates that many patients with UTI infections are likely being underdiagnosed by SUC, especially when the infection is non-E. coli-based or polymicrobial.

### Example 3

The following is a non-limiting example of the present invention. It is to be understood that said example is not intended to limit the present invention in any way. Equivalents or substitutes are within the scope of the present invention.

Urinary tract infections (UTIs) are infections of any part of the urinary tract, generally grouped into lower UTI, called cystitis, in which the infection is confined to the bladder, and upper UTI, called pyelonephritis, in which the infection has spread to the kidneys. UTIs constitute a significant healthcare burden worldwide. A diagnosis of UTI is a leading cause of prescribed antibiotic usage in outpatients, with most infections being treated empirically. Most UTIs occur in otherwise healthy, sexually active, young adult females, in whom anatomic and lifestyle factors result in a predisposition to cystitis. However, while simple UTIs, particularly cystitis, are typically managed successfully with empirically prescribed antibiotics in an outpatient setting, patients with additional risk factors often require guided treatment. Newborns, children, elderly adults, and persons with diabetes or other comorbidities are at increased risk for recurrent and/or complicated UTIs (r/cUTIs). These groups, particularly elderly adults, have higher treatment failure rates and poorer outcomes, such as UTI recurrence, urosepsis, and even death. In 2018 r/cUTIs accounted for approximately >600,000 hospitalizations at an estimated mean cost of $70,063 per hospitalization (non-CAUTI related) in the US. As the threat of microbial antibiotic resistance continues to increase, providing the correct antibiotic treatment quickly enough to avoid prolonged empiric therapy is a growing concern among healthcare stakeholders.

As a diagnostic test for UTI, standard urine culture (SUC) has been in use for over 60 years with little advancement to accommodate for the identification of more recently discovered emerging uropathogens. The standard urine culture method is optimized for the growth of gram-negative bacteria, primarily Escherichia coli (E. coli), the most commonly identified organism in acute UTIs. Furthermore, the turn-around time for SUC, which includes antimicrobial susceptibility testing, can be 3-5 days, potentially delaying results-guided antimicrobial treatment even in cases where the causative organism is detected. Recent studies have shown that when more sensitive culture techniques such as enhanced-quantitative urine culture (EQUC) are used, many additional clinically relevant microbial species including several gram-positive organisms, fastidious microbes, and fungi have been isolated from symptomatic subjects.

Multiplex-PCR (M-PCR) is superior for detecting non-E. coli and polymicrobial infections in urine specimens compared to SUC (see Example 2). Polymicrobial infections, which have been reported in up to 39% of suspected UTI cases in older adult populations, have specifically been associated with poorer outcomes. Additionally, M-PCR has the benefit of faster turnaround times to reported results, allowing for a more rapid transition to directed antimicrobial therapy or avoiding empiric therapy altogether.

The purpose of this study was to validate the relevance of individual microbial species or groups using three infection-associated biomarkers, NGAL, IL-1β, and IL-8, as indicators of the state of the immune system in conjunction with a unique M-PCR assay for detection and quantification of microorganisms in patients with lower urinary tract symptoms diagnosed presumptively with UTIs in a specialty setting.

Study Design: This study utilized banked urine specimens from a randomly collected cross-section of 1132 subjects, at least 60 years old, presenting at urology clinics in 22 US states between Jan. 17, 2023 and May 16, 2023 with clinical presentations consistent with UTI, and for which there was enough specimen to effectively conduct M-PCR and biomarker studies. The samples included in the biobank and used for this analysis are intended to be representative of the samples that would routinely be sent for urine microorganism identification and quantification testing as part of the diagnosis and management of cases seen in outpatient urologic specialty settings. Since this study utilized urine samples from a biobank in which the samples were de-identified and associated only with the assigned ICD-10-CM code(s) and the subject's age and sex, the study was exempted from review from the Western Institutional Review Board-Copernicus Group (WCG), an external independent agency that reviews and approves industry-sponsored clinical trials.

TABLE 11
Biomarker Positivity Cutoffs

| Biomarkers | Cutoff |
|---|---|
| Neutrophil gelatinase-associated lipocalin (NGAL) | ≥38.0 ng/mL |
| Interleukin 8 (IL-8) | ≥20.6 pg/mL |
| Interleukin 1 beta (IL-1β) | ≥12.4 pg/mL |

All urine samples utilized in this study were collected via the midstream voided “clean catch” method which is standard practice for busy clinical offices. Samples were transferred to gray-top boric acid (for M-PCR) and yellow-top (for P-AST and biomarker analysis) Vacutainer Tubes (Becton Dickinson, Franklin Lakes, NJ) and shipped overnight at ambient temperature for evaluation at a central testing laboratory (Pathnostics, Irvine CA). Urine samples were processed for M-PCR/P-AST and for urinary biomarkers (NGAL, IL-1β, and IL-8) by enzyme-linked immunosorbent assay (ELISA). Only samples where microbes were detected above a positivity threshold≥10,000 cells/mL for bacteria/bacterial groups and >0 cells/mL for yeasts by M-PCR were included in the biomarker analysis.

Specimen testing closely resembles the methodology outlined in Example 1.

Subject Demographics: The study included 1132 subjects presenting to urology clinics with symptoms of r/cUTI. The median subject age was 76.3 (range 60.0-103 years), and the mean was 76.6 (standard deviation=8.72). Female patients comprised the majority of the cohort, 66.4% (n=752), and males accounted for 33.6% (n=380) (Table 12). Many specimens were associated with 2 or more ICD-10-CM (https://www.icd10data.com) codes. The most prevalent of these ICD-10-CM codes was N39.0 “Urinary tract infection, site not specified” [76.0% (n=977)]; followed by R30.0 “Dysuria” [8.1% (n=104)]; R31.0 “Gross hematuria” [3.3% (n=42)]; Z87.440 for “Personal history of urinary (tract) infections” [1.8% (n=23)]; and R31.9 for “Hematuria, unspecified” [1.2% (n=16)]. All other r/cUTI-related ICD-10-CM codes, each with a prevalence of <1% of subjects, were grouped under “Other” (Table 13).

TABLE 12
Demographic of the Study Cohort
Demographics

| | | | | | |
|---|---|---|---|---|---|
| Age | Mean (SD) | 76.6 (8.72) | Sex | Female | 752 (66.4%) |
| | Median [Min, | 76.3 [60.0, | | Male | 380 (33.6%) |
| | Max] | 103] | | | |
| | | | | Total | 1132 |

TABLE 13
Summary of Most Prevalent ICD-10-CM
Codes for the Study Cohort

| ICD-10-CM Code | Code Description | Prevalence |
|---|---|---|
| N39.0 | Urinary tract infection, site | 76.0% (n = 977) |
| | not specified | |
| R30.0 | Dysuria | 8.1% (n = 104) |
| R31.0 | Gross hematuria | 3.3% (n = 42) |
| Z87.440 | Personal history of diseases of | 1.8% (n = 23) |
| | urinary system | |
| R31.9 | Hematuria, unspecified | 1.2% (n = 16) |
| Others | | 9.5% (n = 122) |

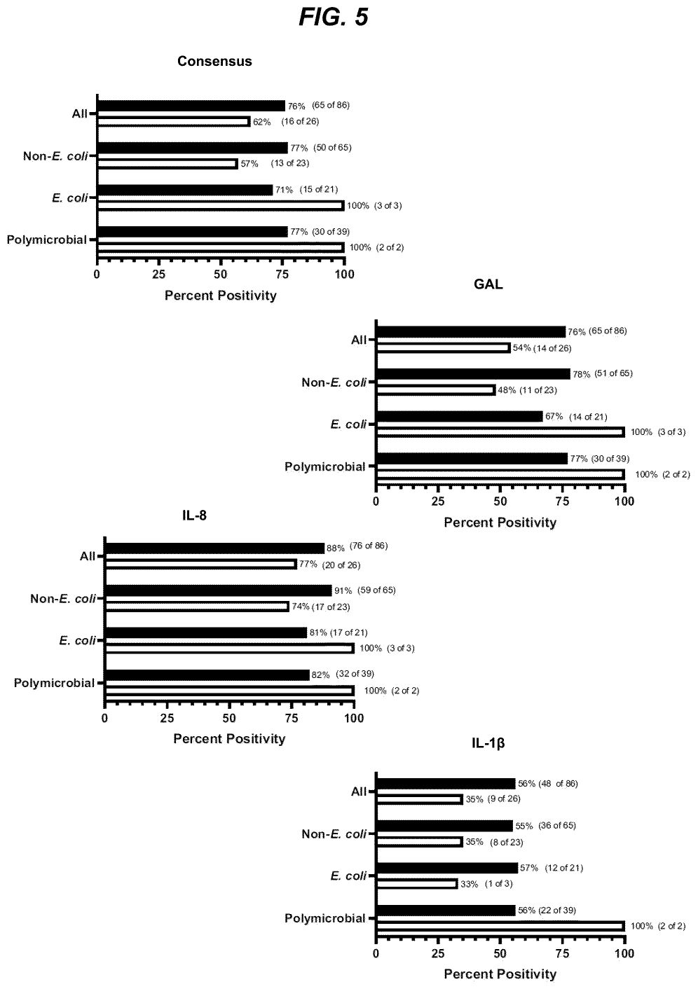

Microbial Prevalence with Detection and Identification by M-PCR/P-AST Assay: All 1132 specimens were tested for the presence of microbes by M-PCR; of those, 823 (72.7%) were positive. Within these positive specimens, M-PCR identified 1589 microorganisms, with a significant fraction of the total cases being polymicrobial infections (n=522, 46.1%). Of the 27 species and three groups of microorganisms included in the M-PCR assay, only two (A. baumannii and P. agglomerans) were not detected in any specimen (FIG. 6). Two-thirds of the microorganisms (20 of 30) accounted for approximately 99% of all positive results at the case level (Table 14).

TABLE 14

| | M-PCR | Biomarker Positive | | | |
|---|---|---|---|---|---|
| Organisms as Detected at | Positive | Consensus | NGAL | IL-8 | IL-1β |
| Density ≥10,000 by M-PCR | n (%) | n (%) | n (%) | n (%) | n (%) |
| Overall | 823 | 661 | 670 | 709 | 529 |
| | (73%) | (80%) | (81%) | (86%) | (64%) |
| Top 5 (Escherichia coli, Aerococcus | 648 | 517 | 526 | 556 | 415 |
| urinae, Actinobaculum schaalii, | (79%) | (80%) | (81%) | (86%) | (64%) |
| Enterococcus faecalis, Viridans Group | | | | | |
| Streptococcus) | | | | | |
| Top 10 (Escherichia coli, Aerococcus | 763 | 609 | 617 | 655 | 491 |
| urinae, Actinobaculum schaalii, | (93%) | (80%) | (81%) | (86%) | (64%) |
| Enterococcus faecalis, Viridans Group | | | | | |
| Streptococcus, Klebsiella pneumoniae, | | | | | |
| Gardnerella vaginalis, Coagulase | | | | | |
| Negative Staphylococcus, | | | | | |
| Pseudomonas aeruginosa, Proteus | | | | | |
| mirabilis) | | | | | |
| Top 15 (Escherichia coli, Aerococcus | 800 | 641 | 648 | 688 | 512 |
| urinae, Actinobaculum schaalii, | (97%) | (80%) | (81%) | (86%) | (64%) |
| Enterococcus faecalis, Viridans Group | | | | | |
| Streptococcus, Klebsiella pneumoniae, | | | | | |
| Gardnerella vaginalis, Coagulase | | | | | |
| Negative Staphylococcus, | | | | | |
| Pseudomonas aeruginosa, Proteus | | | | | |
| mirabilis, Alloscardovia omnicolens, | | | | | |
| Enterobacter Group, Streptococcus | | | | | |
| agalactiae, Candida glabrata, | | | | | |
| Enterococcus faecium) | | | | | |
| Top 20 (Escherichia coli, Aerococcus | 816 | 655 | 664 | 702 | 523 |
| urinae, Actinobaculum schaalii, | (99%) | (80%) | (81%) | (86%) | (64%) |
| Enterococcus faecalis, Viridans Group | | | | | |
| Streptococcus, Klebsiella pneumoniae, | | | | | |
| Gardnerella vaginalis, Coagulase | | | | | |
| Negative Staphylococcus, | | | | | |
| Pseudomonas aeruginosa, Proteus | | | | | |
| mirabilis, Alloscardovia omnicolens, | | | | | |
| Enterobacter Group, Streptococcus | | | | | |
| agalactiae, Candida glabrata, | | | | | |
| Enterococcus faecium, Candida | | | | | |
| albicans, Ureaplasma urealyticum, | | | | | |
| Klebsiella oxytoca, Morganella morganii, | | | | | |
| Citrobacter freundii) | | | | | |
| Top 25 (Escherichia coli, Aerococcus | 822 | 661 | 670 | 708 | 529 |
| urinae, Actinobaculum schaalii, | (100%) | (80%) | (82%) | (86%) | (64%) |
| Enterococcus faecalis, Viridans Group | | | | | |
| Streptococcus, Klebsiella pneumoniae, | | | | | |
| Gardnerella vaginalis, Coagulase | | | | | |
| Negative Staphylococcus, | | | | | |
| Pseudomonas aeruginosa, Proteus | | | | | |
| mirabilis, Alloscardovia omnicolens, | | | | | |
| Enterobacter Group, Streptococcus | | | | | |
| agalactiae, Candida glabrata, | | | | | |
| Enterococcus faecium, Candida | | | | | |
| albicans, Ureaplasma urealyticum, | | | | | |
| Klebsiella oxytoca, Morganella morganii, | | | | | |
| Citrobacter freundii, Corynebacterium | | | | | |
| riegelii, Staphylococcus aureus, | | | | | |
| Mycoplasma hominis, Citrobacter | | | | | |
| koseri, Candida parapsilosis) | | | | | |
| Top 30 (Escherichia coli, Aerococcus | 823 | 661 | 670 | 709 | 529 |
| urinae, Actinobaculum schaalii, | (100%) | (80%) | (81%) | (86%) | (64%) |
| Enterococcus faecalis, Viridans Group | | | | | |
| Streptococcus, Klebsiella pneumoniae, | | | | | |
| Gardnerella vaginalis, Coagulase | | | | | |
| Negative Staphylococcus, | | | | | |
| Pseudomonas aeruginosa, Proteus | | | | | |
| mirabilis, Alloscardovia omnicolens, | | | | | |
| Enterobacter Group, Streptococcus | | | | | |
| agalactiae, Candida glabrata, | | | | | |
| Enterococcus faecium, Candida | | | | | |
| albicans, Ureaplasma urealyticum, | | | | | |
| Klebsiella oxytoca, Morganella morganii, | | | | | |
| Citrobacter freundii, Corynebacterium | | | | | |
| riegelii, Staphylococcus aureus, | | | | | |
| Mycoplasma hominis, Citrobacter | | | | | |
| koseri, Candida parapsilosis, Serratia | | | | | |
| marcescens, Candida auris, Providencia | | | | | |
| stuartii, Acinetobacter baumannii, | | | | | |
| Pantoea agglomerans) | | | | | |

The levels of biomarkers were analyzed based on the classification groups of the detected microorganisms. The list of classifications and references is provided in Table 15. Among the top five most prevalent organisms, a diverse representation was observed: one belonged to the classical gram-negative category (E. coli), one to the classical gram-positive type (E. faecalis), and three belonged to the emerging and/or fastidious uropathogen group (A. urinae, A. schaalii, and Viridans Group Streptococcus [VGS]). Gram-negative bacteria were detected in 581 (51.3%) specimens with over half of those (57.8%, n=336) identified as E. coli. Gram-positive bacteria were detected in 438 (38.7%) specimens, of which 40.4% (n=177) were identified as E. faecalis. Fastidious organisms were detected in 570 (50.4%) of total cases. A. urinae was the predominant species identified in 224 (39.3%) cases with fastidious organisms detected. Yeasts were detected in 40 cases (3.5%), and C. glabrata accounted for over half of the detected yeasts (n=22, 55%). Additionally, two organisms traditionally considered contaminants from the skin, VGS [(n=160), 14.1% and Coagulase Negative Staphylococcus (CoNS) [(n=49), 4.3%], were among the top 10 most prevalent organisms detected in the study specimens.

TABLE 15

| | Characteristics | |
|---|---|---|
| | (gram-negative, | Classification |
| | gram-positive, | (Classical or |
| Microorganism | fastidious, yeast) | Emerging) |
| Acinetobacter baumannii* | gram-negative | emerging |
| Actinotignum schaalii | fastidious | emerging |
| Aerococcus urinae | fastidious | emerging |
| Alloscardovia omnicolens | fastidious | emerging |
| Candida albicans | yeast | classical |
| Candida auris | yeast | classical |
| Candida glabrata | yeast | classical |
| Candida parapsilosis | yeast | classical |
| Citrobacter freundii* | gram-negative | classical |
| Citrobacter koseri* | gram-negative | classical |
| Corynebacterium riegelii | fastidious | emerging |
| Enterococcus faecalis* | gram-positive | classical |
| Enterococcus faecium | gram-positive | classical |
| Escherichia coli* | gram-negative | classical |
| Gardnerella vaginalis | fastidious | emerging |
| Klebsiella oxytoca* | gram-negative | classical |
| Klebsiella pneumoniae* | gram-negative | classical |
| Morganella morganii* | gram-negative | classical |
| Mycoplasma hominis | fastidious | emerging |
| Pantoea agglomerans* | gram-negative | classical |
| Proteus mirabilis* | gram-negative | classical |
| Providencia stuartii* | gram-negative | classical |
| Pseudomonas aeruginosa* | gram-negative | classical |
| Serratia marcescens* | gram-negative | classical |
| Staphylococcus aureus* | gram-positive | classical |
| Streptococcus agalactiae* | gram-positive | classical |
| Ureaplasma urealyticum | fastidious | emerging |
| Coagulase-negative Staphylococcus | gram-positive | emerging |
| (CoNS)* | | |
| Viridans group Streptococcus | gram-positive | emerging |
| Enterobacter Group* | gram-negative | classical |

CoNS includes Staphylococcus epidermidis, Staphylococcus haemolyticus, Staphylococcus lugdunenesis, and Staphylococcus saprophyticus; VGS includes Streptococcus anginosus, Streptococcus oralis, and Streptococcus pasteuranus; Enterobacter Group includes Klebsiella aerogenes (formally known as Enterobacter aerogenes), and Enterobacter cloacae

Infection-Associated Biomarkers in M-PCR-Positive Urine Samples: In order to comprehensively assess the presence of infection-associated biomarkers (NGAL, IL-8, and IL-1β), the same urine specimens in which microorganisms were detected by M-PCR were analyzed. By comparing biomarker positivity based on the thresholds outlined in Table 11, the rate of biomarker positivity was examined among different groups of organisms. In Table 16, the 30 detectable microorganisms are presented in groups of 5, by descending order of prevalence, starting with the five most frequently detected organisms, followed by the next most prevalent 5, and ending with the five organisms detected with the least frequency.

TABLE 16
Biomarker Positivity in Groups of Five Organisms by Prevalence

| | | Individual and Consensus Biomarker Positivity | | | |
|---|---|---|---|---|---|
| | M-PCR | Among All M-PCR- Positive Cases | | | |
| | Positivity | Consensus | NGAL | IL-8 | IL-1β |
| | n (%) of | n (%) of | n (%) of | n (%) of | n (%) of |
| Organisms as Detected at | M- PCR | M-PCR | M-PCR | M-PCR | M-PCR |
| Density ≥10,000 by M-PCR, | Positive | Positive | Positive | Positive | Positive |
| in Order of Prevalence | Cases | Cases | Cases | Cases | Cases |
| Overall M-PCR Positive Cases | 823 | 661 | 670 | 709 | 529 |
| | (100%) | (80%) | (81%) | (86%) | (64%) |
| 5 Most Prevalent Organisms | 648 | 517 | 526 | 556 | 415 |
| (Escherichia coli, Aerococcus urinae, | (79%) | (80%) | (81%) | (86%) | (64%) |
| Actinobaculum schaalii, Enterococcus | | | | | |
| faecalis, Viridans Group | | | | | |
| Streptococcus) | | | | | |
| Next 5 (Klebsiella pneumoniae, | 272 | 226 | 228 | 240 | 181 |
| Gardnerella vaginalis, Coagulase | (33%) | (83%) | (84%) | (88%) | (67%) |
| Negative Staphylococcus, | | | | | |
| Pseudomonas aeruginosa, Proteus | | | | | |
| mirabilis) | | | | | |
| Next 5 (Alloscardovia omnicolens, | 117 | 98 | 98 | 101 | 74 |
| Enterobacter group, Streptococcus | (14%) | (84%) | (84%) | (86%) | (63%) |
| agalactiae, Candida glabrata, | | | | | |
| Enterococcus faecium) | | | | | |
| Next 5 (Candida albicans, | 60 | 54 | 54 | 54 | 46 |
| Ureaplasma urealyticum, Klebsiella | (7%) | (90%) | (90%) | (90%) | (77%) |
| oxytoca, Morganella morganii, | | | | | |
| Citrobacter freundii) | | | | | |
| Next 5 (Corynebacterium riegelii, | 26 | 24 | 25 | 24 | 18 |
| Staphylococcus aureus, Mycoplasma | (3%) | (92%) | (96%) | (92%) | (69%) |
| hominis, Citrobacter koseri, Candida | | | | | |
| parapsilosis) | | | | | |
| 5 Least Prevalent Organisms | 4 | 2 | 2 | 4 | 2 |
| (Serratia marcescens, Candida auris, | (<1%) | (50%) | (50%) | (100%) | (50%) |
| Providencia stuartii, Acinetobacter | | | | | |
| baumannii, Pantoea agglomeran | | | | | |

Notes:
Biomarker positivity is presented by the total number and percentage of multiplex polymerase chain reaction (M-PCR) positives for each microbe-prevalence group.

Urine samples with detected organisms exhibited high percentages of biomarker positivity. Specifically, NGAL showed a positivity rate of 81%, IL-8 showed a positivity rate of 86%, and IL-1β exhibited a positivity rate of 64%. Furthermore, the simultaneous positivity rate of two or more biomarkers was observed in 80% of cases. To provide a more granular analysis, we delved into the biomarker positivity rates for each individual organism (refer to Table 17) and sub-grouped the organisms starting with the top five most detected organisms, gradually expanding in groups of five (refer to Table 14).

TABLE 17

| Organisms as Detected | M-PCR | | Biomarker Positive | | | | | | | |
|---|---|---|---|---|---|---|---|---|---|---|
| at Density ≥10,000 | Positive | | Consensus | | NGAL | | IL-8 | | IL-1β | |
| by M-PCR | n (%) | | n (%) | | n (%) | | n (%) | | n (%) | |
| Overall | 823 | (73%) | 661 | (80%) | 670 | (81%) | 709 | (86%) | 529 | (64%) |
| Escherichia coli | 336 | (41%) | 289 | (86%) | 289 | (86%) | 305 | (91%) | 243 | (72%) |
| Aerococcus urinae | 224 | (27%) | 180 | (80%) | 192 | (86%) | 193 | (86%) | 142 | (63%) |
| Actinobaculum schaalii | 222 | (27%) | 182 | (82%) | 196 | (88%) | 193 | (87%) | 140 | (63%) |
| Enterococcus faecalis | 177 | (22%) | 134 | (76%) | 134 | (76%) | 153 | (86%) | 103 | (58%) |
| Viridans Group | 160 | (19%) | 120 | (75%) | 125 | (78%) | 130 | (81%) | 90 | (56%) |
| Streptococcus | | | | | | | | | | |
| Klebsiella pneumoniae | 111 | (13%) | 97 | (87%) | 96 | (86%) | 101 | (91%) | 76 | (68%) |
| Gardnerella vaginalis | 71 | (9%) | 47 | (66%) | 48 | (68%) | 57 | (80%) | 29 | (41%) |
| Coagulase Negative | 49 | (6%) | 41 | (84%) | 45 | (92%) | 42 | (86%) | 36 | (73%) |
| Staphylococcus | | | | | | | | | | |
| Pseudomonas | 35 | (4%) | 33 | (94%) | 34 | (97%) | 33 | (94%) | 29 | (83%) |
| aeruginosa | | | | | | | | | | |
| Proteus mirabilis | 34 | (4%) | 32 | (94%) | 30 | (88%) | 33 | (97%) | 26 | (76%) |
| Alloscardovia omnicolens | 27 | (3%) | 21 | (78%) | 20 | (74%) | 22 | (81%) | 13 | (48%) |
| Enterobacter Group | 26 | (3%) | 23 | (88%) | 22 | (85%) | 24 | (92%) | 19 | (73%) |
| Streptococcus agalactiae | 24 | (3%) | 20 | (83%) | 20 | (83%) | 20 | (83%) | 17 | (71%) |
| Candida glabrata | 22 | (3%) | 19 | (86%) | 20 | (91%) | 19 | (86%) | 18 | (82%) |
| Enterococcus faecium | 20 | (2%) | 17 | (85%) | 18 | (90%) | 18 | (90%) | 9 | (45%) |
| Candida albicans | 15 | (2%) | 13 | (87%) | 14 | (93%) | 13 | (87%) | 11 | (73%) |
| Ureaplasma urealyticum | 13 | (2%) | 11 | (85%) | 10 | (77%) | 12 | (92%) | 9 | (69%) |
| Klebsiella oxytoca | 12 | (1%) | 11 | (92%) | 11 | (92%) | 11 | (92%) | 11 | (92%) |
| Morganella morganii | 11 | (1%) | 11 | (100%) | 11 | (100%) | 11 | (100%) | 9 | (82%) |
| Citrobacter freundii | 10 | (1%) | 9 | (90%) | 9 | (90%) | 8 | (80%) | 7 | (70%) |
| Corynebacterium riegelii | 8 | (1%) | 7 | (88%) | 8 | (100%) | 7 | (88%) | 5 | (62%) |
| Staphylococcus aureus | 8 | (1%) | 7 | (88%) | 7 | (88%) | 8 | (100%) | 4 | (50%) |
| Mycoplasma hominis | 5 | (1%) | 5 | (100%) | 5 | (100%) | 4 | (80%) | 5 | (100%) |
| Citrobacter koseri | 3 | (0%) | 3 | (100%) | 3 | (100%) | 3 | (100%) | 3 | (100%) |
| Candida parapsilosis | 2 | (0%) | 2 | (100%) | 2 | (100%) | 2 | (100%) | 1 | (50%) |
| Serratia marcescens | 2 | (0%) | 1 | (50%) | 1 | (50%) | 2 | (100%) | 1 | (50%) |
| Candida auris | 1 | (0%) | 1 | (100%) | 1 | (100%) | 1 | (100%) | 1 | (100%) |
| Providencia stuartii | 1 | (0%) | 0 | (0%) | 0 | (0%) | 1 | (100%) | 0 | (0%) |
| Acinetobacter baumannii | 0 | (0%) | 0 | (NA) | 0 | (NA) | 0 | (NA) | 0 | (NA) |
| Pantoea agglomerans | 0 | (0%) | 0 | (NA) | 0 | (NA) | 0 | (NA) | 0 | (NA) |

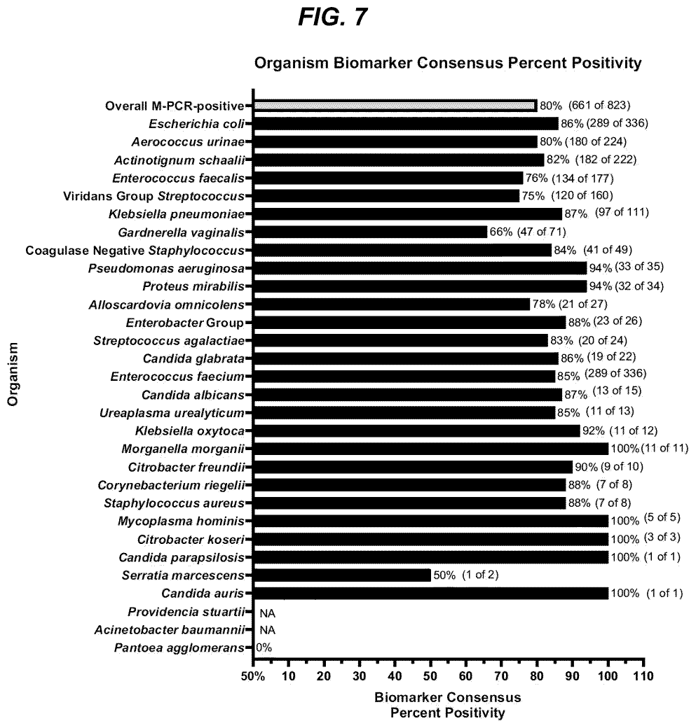

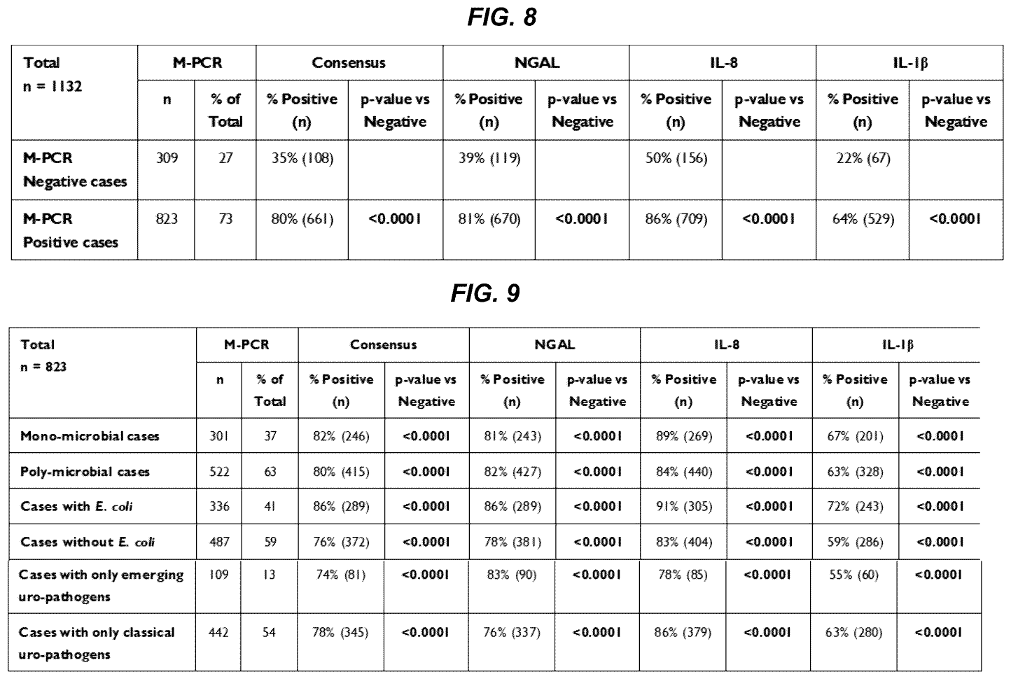

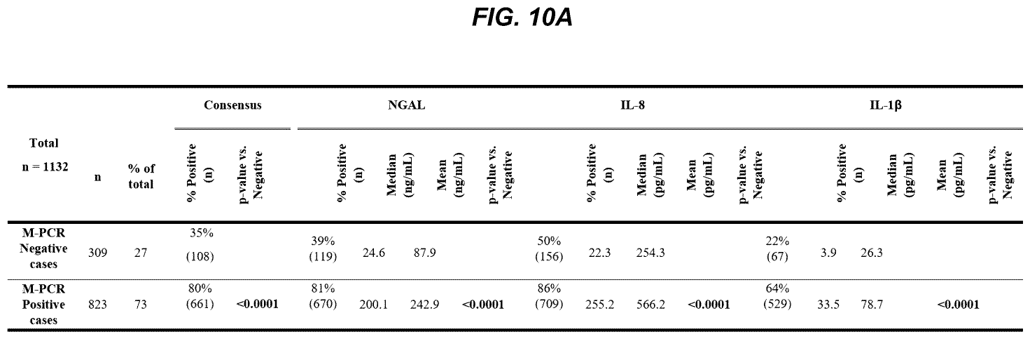

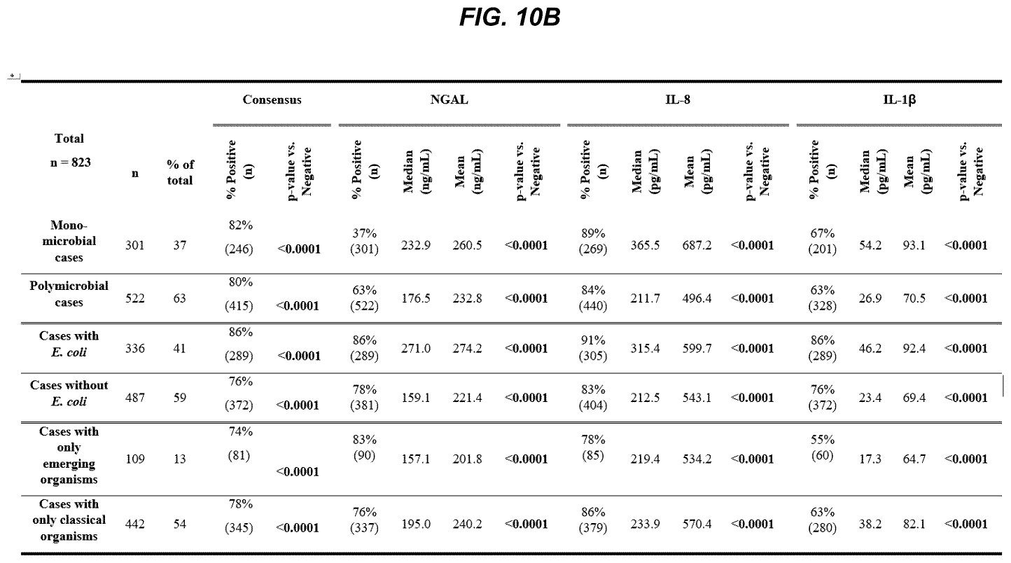

Considering the remarkable sensitivity of M-PCR in detecting a diverse array of organisms extending beyond E. coli and classical uropathogens, the biomarker positivity was examined in all (both positive and negative) M-PCR specimens (FIG. 8 and FIG. 10A) and stratified cases into different groups (FIG. 9 and FIG. 10B). These groups comprised cases with and without E. coli detection, cases with solely classical uropathogens detected, and cases exhibiting exclusively emerging uropathogens (FIG. 9 and FIG. 10B). Furthermore, given the substantial capability of M-PCR to identify a significantly higher number of polymicrobial infections we also compared biomarker levels between specimens with monomicrobial and polymicrobial samples (FIG. 9, and FIG. 10B). 12,13,18,19 Across all groups of M-PCR positive specimens, all three biomarkers (NGAL, IL-8, and IL-1β) had a significantly higher percent positivity (p<0.0001) than in M-PCR negative cases.

The biomarker consensus percent positivity for each subgroup (FIG. 9) was examined. All subgroups of M-PCR-positive specimens had a biomarker consensus positivity>70%, ranging from 74% for cases with only emerging uropathogens to 86% for cases with E. coli detected. The biomarker consensus positivity for each subgroup was also significantly higher than that of the M-PCR-negative cases (p<0.0001).

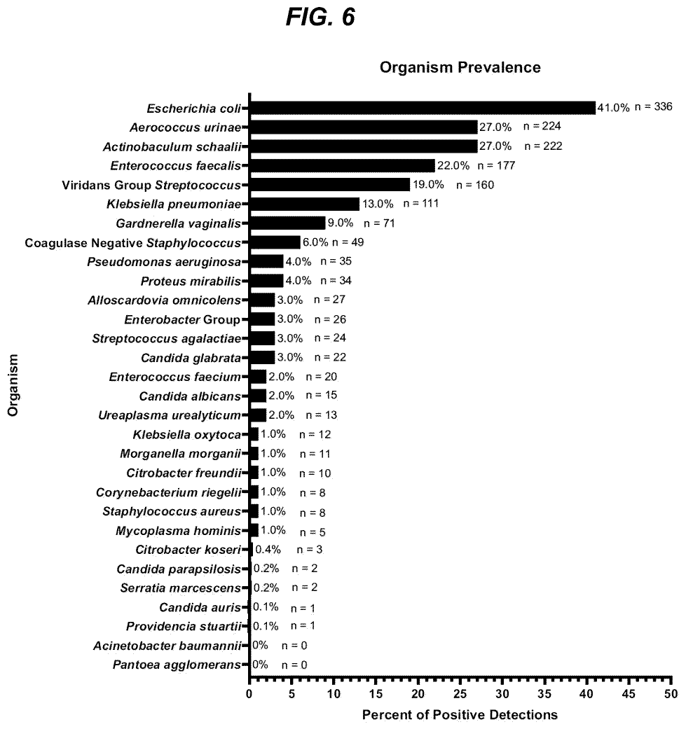

The biomarker consensus positivity was also examined for individual organisms to confirm their status as uropathogenic organisms (FIG. 7). Biomarker consensus positivity rates were independent of microorganism prevalence in the study cohort. For example, Gardnerella vaginalis, the seventh most prevalent microorganism detected (n=71) had a 66% biomarker consensus percent positivity, while Mycoplasma hominis, detected in only 5 specimens, had 100% biomarker consensus percent positivity (FIG. 7 and Table 17). Of the detected organisms, all but 2 [Providencia stuartii (n=1) and Serratia marcescens (n=2)] had >66% consensus biomarker positivity. Overall, 80% (n=661) of the 823 M-PCR-positive specimens were positive for biomarker consensus.

To guide antimicrobial selection for UTI patients, clinicians currently rely on microbial identification and quantitation by SUC and associated antibiotic susceptibility testing. Failure to detect and correctly identify those organisms missed by SUC can result in many UTIs remaining undiagnosed and untreated or being sub-optimally treated with empiric broad-spectrum antibiotics which potentially prolongs symptoms or results in serious complications. Though it is evident that M-PCR has greater sensitivity than SUC, there are some questions about the value of detecting these organisms, and whether they are associated with UTIs or are incidental findings.

Of the 30 organisms/organism groups included in the assay for this study just two (A. baumannii and P. agglomerans) were not detected in these symptomatic presumed UTI cases, though those 2 organisms have previously been shown to be uropathogenic. Additionally, two organisms traditionally considered contaminants from skin, VGS and CONS, were among the top 10 most prevalent organisms detected in the study specimens. Other studies identified VGS and CoNS in both midstream voided and catheter-collected specimens at similar prevalence and densities, further indicating the organisms' likely pathogenic nature. Therefore, the approach of detecting only classical uropathogens may result in missed cases, as emerging or less common pathogens can cause infections and pose significant health threats. Having shown that these organisms were found within a significant number of presumed UTI cases, their association with urinary biomarkers associated with UTIs was then examined. Biomarker percent positivity was significantly higher for all 3 biomarkers in M-PCR positive specimens, compared to M-PCR negatives overall (p<0.001). The small number of M-PCR-negative specimens with elevated biomarkers may represent UTIs caused by viruses, yeast, or bacteria not targeted by the M-PCR test, or by non-infectious bladder inflammation, such as interstitial cystitis.

Using biomarker consensus percent positivity, whether the detection of a spectrum of organisms present in this assay was associated with positive consensus biomarkers was then examined. Overall, 80% of M-PCR-positive specimens were positive for biomarker consensus. Further, of the 28 detected organisms, all but two [P. stuartii (n=1) and S. marcescens (n=2)] had >66% of cases with consensus biomarker positivity. When the organisms were categorized into groups of 5 by decreasing prevalence, all groups showed an association with positive consensus and individual markers. Polymicrobial cases, monomicrobial cases, cases with only classical uropathogens, cases with only emerging uropathogens, cases with E. coli, and those without E. coli all had elevated biomarkers. These results strongly indicate that the microbes detected by this assay, many of which are fastidious and emerging pathogens that will likely be missed by SUC, are causative of the UTIs in these cases and not incidental findings. These results make it important to question whether SUC is underdiagnosing due to low sensitivity when the clinical diagnosis from a urology specialty setting, and the urine inflammatory biomarkers agree a UTI is present.

A subset of specimens exhibited outlier data points with low inflammatory biomarker levels despite high microbial densities detected by M-PCR. These outliers with low inflammatory biomarker levels may reflect scenarios where immune responses are compromised due to medications or underlying health conditions, or instances of a resolving UTI.

This example shows symptomatic cases involving non-E. coli and emerging uropathogens, along with VGS and CoNS were associated with substantially higher levels of all three biomarkers than in cases where no organisms were detected. These findings suggest that the assay size should not be limited to E. coli and a small set of highly prevalent and longstanding uropathogens, which would lead to missed UTI diagnosis and potentially poorer treatment outcomes. Almost all of the organisms present here would be important to include in a UTI assay in order to be confident that most UTI cases could have the causative pathogen identified.

The biggest strength of this study was the comparison of the immune response according to biomarkers NGAL, IL-1β, and IL-8 in a large number of samples obtained from UTI symptomatic patients from a urology specialty setting, against the identification of microorganisms detected by M-PCR from the same urine sample. This approach allowed us to directly associate the presence of microorganisms identified by the M-PCR assay to infection-associated immune responses in the urinary tract of each subject.

Measuring microbial detection by M-PCR against positivity rates of three UTI-associated biomarkers (NGAL, IL-8, and IL-1β), whether these 30 microorganisms were clinically relevant for UTI diagnostics was determined. In the study cohort of 1132 individuals>60 years of age who were symptomatic for r/cUTI, 28 of 30 microorganisms included in the M-PCR assay were detected. Additionally, 80% of M-PCR positive specimens were positive for biomarker consensus, which in symptomatic patients diagnosed in a specialty setting are a measure of inflammation with high sensitivity and specificity for UTI. Together, these findings demonstrate that the majority of organisms detected by the M-PCR assay are likely clinically relevant with high specificity and that their detection by M-PCR has a low likelihood of false-positivity for UTI.

### Example 4

The following is a non-limiting example of the present invention. It is to be understood that said example is not intended to limit the present invention in any way. Equivalents or substitutes are within the scope of the present invention.

Study Design and Participants: Results from biomarker analyses, M-PCR/P-AST tests, and standard urine culture (SUC) included in this analysis were obtained from urine samples from two clinical studies: One was a prospective observational study (WCG IRB 20230847) that enrolled subjects 60 years of age or older who were asymptomatic for UTI. Subjects were recruited from the community (at theaters, sporting events, social gatherings, etc.) and provided written informed consent prior to enrollment. Subjects who were pregnant, taking antibiotics for a UTI, or who had cancer of the urinary tract were excluded. A total of 228 asymptomatic subjects from two states were enrolled in the study between Feb. 28, 2023 and Mar. 22, 2023. All subjects in the study completed the validated American English Acute Cystitis Symptom Score (ACSS) baseline questionnaire and a short medical history and provided a midstream voided urine specimen. Symptom status was determined using the US Food and Drug Administration (FDA) symptom scores on the validated American English Acute Cystitis Symptom Score (ACSS) Questionnaire, asking patients to evaluate four typical UTI symptoms: urinary frequency, urinary urgency, dysuria, and suprapubic pain, as well as visible blood in the urine, according to each one's severity (scoring 0-3): no (0), mild (1), moderate (2), severe (3). Asymptomatic cases were defined as having four FDA symptom scores adding up to <4, none of the four symptom scores being >1, and the absence of visible blood in the urine.

The other was a biorepository study from which the symptomatic cohort samples were obtained. Urine samples from patients 60 years of age or older who presented to outpatient urology clinics in 39 states with symptom(s) and ICD-10-CM codes consistent with UTI were collected, de-identified, and stored into the biorepository bank with 583 urine samples accrued in the bank between Jan. 17, 2023 and Apr. 24, 2023. Each de-identified urine sample was assigned a repository label associated with a record of the subject's age, sex, and ICD-10-CM code(s) and stored in a biorepository for evaluation at Pathnostics' clinical laboratory. The WCG IRB deemed the biorepository-obtained specimens exempt from review under 45 CFR § 46.104(d)(4), as data from the study was collected via a deidentified database and used in a manner that the identity of the subject cannot be readily ascertained directly or through identifiers linked to the subjects, and that the investigator would not contact or re-identify the subjects.

Urine specimens from both studies were collected via the midstream clean-catch/voided method. Results from biomarker analyses, M-PCR/P-AST, and SUC per-formed side by side from the urine samples from these 228 asymptomatic subjects and 583 symptomatic subjects were analyzed to investigate if infection-associated urine biomarkers can differentiate definitive UTIs from non-UTI controls.

The Guidance® UTI M-PCR/P-AST assay: The test includes susceptibility testing for 19 antibiotics, semi-quantification of 27 distinct uropathogenic species and three bacterial groups, as well as identification of 32 antibiotic-resistance genes and the ESBL phenotype. The test was performed as described previously: the first step involves DNA extraction from the subject's urine sample using KingFisher/MagMAX™ automated DNA extraction instrument and the MagMAX™ DNA Multi-Sample Ultra Kit (Thermo Fisher Scientific, Carlsbad, CA) per the manufacturer's instructions. Extracted DNA was mixed with a universal PCR master mix and amplified using TaqMan technology in a Life Technologies 12 K Flex 112-format OpenArray System (Thermo Fisher Scientific, Wilmington, NC). Probes and primers were used to detect 23 bacterial species and 3 bacterial groups, fastidious and nonfastidious, and four yeast species listed below:

Classical uropathogens: Candida albicans, Candida glabrata, Candida parapsilosis, Citrobacter freundii, Citrobacter koseri, Enterococcus faecalis, Enterococcus faecium, Escherichia coli, Klebsiella oxytoca, Klebsiella pneumoniae, Morganella morganii, Pantoea agglomer-ans, Proteus mirabilis, Providencia stuartii, Pseudomonas aeruginosa, Serratia marcescens, Staphylococcus aureus, Streptococcus agalactiae, and Enterobacter group [including Klebsiella aerogenes (formally known as Enterobacter aerogenes) and Enterobacter cloacae].

Emerging uropathogens: Acinetobacter baumannii, Actinotignum schaalii, Aerococcus urinae, Alloscardovia omnicolens, Candida auris, Corynebacterium riegelii, Gardnerella vaginalis, Mycoplasma hominis, Ureaplasma urealyticum, coagulase-negative staphylococci group (CoNS) (including Staphylococcus epidermidis, Staphylococcus haemolyticus, Staphylococcus lugdunesis, and Staphylococcus saprophyticus), and Viridans group streptococci (VGS) (including Streptococcus anginosus, Streptococcus oralis, and Streptococcus pasteuranus).

Results of the P-AST portion of the test, a pooled antibiotic susceptibility assay which accounts for bacterial interactions, were not included in this analysis.

Standard urine culture (SUC): The SUC method was performed as previously described. Briefly, urine was vortexed, and a sterile plastic loop (1 μL) was used to inoculate blood agar plates. A sterile plastic loop (1 L) was used also to inoculate colistin and nalidixic acid agar/MacConkey agar (CNA/MAC) plates, one loop-full of urine on the CNA side of the plate and another full loop-full on the MAC side of the plate. All plates were incubated at 35° C. in 5% CO2 for ≥18 h and then examined for evidence of growth. Per standard operating procedures plates with <10,000 CFU/mL were reported as normal urogenital flora [19]. For plates with growth (≥10,000 CFU/mL), the quantity and morphology of each organism were recorded. The maximum readable colony count using the 1 μL loop is >100,000 CFU/mL. Colony counts were performed on blood agar plates. Species identification and colony counts were performed on CNA/MAC plates. Pathogen identification was confirmed with the VITEK 2 Compact System (bioMerieux, Durham, NC).

Enzyme-linked immunosorbent assay (ELISA): Urine levels of NGAL, IL-8, and IL-1β were analyzed according to the manufacturer's instructions, using ELISA kits from R&D Systems/Bio-Techne (Minneapolis, MN), including human Lipocalin-2/NGAL Quantikine ELISA Kit (Catalog number SLCN20), human IL-8/CXCL8 Quantikine ELISA Kit (Catalog number S8000C), and human IL-1β/IL-1F2 Quantikine ELISA kit (Catalog number SLB50). OD readings at 450 and 540 nm, respectively, were measured on an Infinite M Nano+ microplate reader (TECAN, Switzerland).

Statistical analysis: Participant demographics and ICD-10-CM code breakdown were described by summary statistics (e.g., mean and standard deviation (SD) for continuous variables such as age, count, and percentage for categorical vari-ables such as sex and ICD-10-CM code). To evaluate the ability of the biomarkers to differentiate UTI from non-UTI conditions such as asymptomatic bacteriuria, we defined “Definitive UTI cases” and “Definitive non-UTI cases.” Definitive UTI cases were defined using the current standard of care diagnostic criteria of symptoms/clinical presentation by urology/urogynecology specialists combined with the presence of microorganisms in the urine above a certain density threshold and being positive by both SUC and M-PCR (“Both Detected”). Definitive non-UTI cases were defined as asymptomatic subjects regardless of the presence of detectable microbes in the urine.

After conducting a comprehensive power analysis, the results demonstrate that with a sample size of 351 cases of definitive Urinary Tract Infection (UTI) and 228 cases of definitive non-UTI, we can reliably detect effect sizes as small as 0.24 (Cohen's d). This analysis was performed considering an 80% statistical power and a significance level of 0.05. This indicates a solid capability to identify subtle differences between the two groups, with a minimal risk of false positives.

Although 100,000 CFUs/mL by SUC is typically considered diagnostically significant in the US, clinical reviews and guidelines, as well as the data suggest a microbial density threshold of 10,000 cells/mL or CFUs/mL is more clinically relevant (see Example 1). Thus, analyses we performed using both microbial density thresholds of positivity: Criterion 1 (10,000 cells/mL by M-PCR or CFUs/mL by SUC) and Criterion 2 (100,000 cells/mL by M-PCR or CFUs/mL by SUC).

### Criterion 1 Definitions

Definitive UTI cases: Symptomatic cases where M-PCR detected bacterial counts of ≥10,000 or yeast counts >0 cells/mL and SUC detected bacterial counts of ≥10,000 or yeast counts >0 CFUs/mL.

Definitive non-UTI cases: All asymptomatic cases regardless of microbe identification and density.

### Criterion 2 Definitions

Definitive UTI cases: Symptomatic cases where M-PCR detected bacterial counts of ≥100,000 or yeast counts >0 cells/mL and SUC detected bacterial counts of ≥100,000 or yeast counts >0 CFUs/mL.

Asymptomatic cohort: All asymptomatic cases regardless of microbe identifica-tion and density.

Biomarker thresholds were used to determine positive and negative results for the biomarkers (Table 11). Consensus biomarker positivity was defined as >2 of the 3 biomarkers measuring at or above their respective cutoff values. A probit regression was fitted and plotted to describe the relationship between the density of organisms detected and the positivity (proportion of samples from symptomatic and asymptomatic cohorts with biomarker levels above the threshold) for each biomarker. Statistical analysis between sensitivity of different individuals or combinations of biomarkers used a Proportion Z-test. Statistical difference was defined as p<0.05. The confidence intervals of the biomarker clinical performance characteristics (sensitivity, specificity, positive predictive value, negative predictive value, accuracy, positive likelihood ratio, and negative likelihood ratio) were calculated using the exact method.

All the statistical analyses were performed using R 4.2.2

Demographics: A total of 811 unique subjects' urine specimens, 583 from the symptomatic cohort and 228 from the asymptomatic cohort were analyzed. The subjects in the symptomatic cohort trended slightly older [mean 76.6, median 76.3, range 60.0-99.0 years] than subjects in the asymptomatic cohort [mean 68.8, median 67.5 years, range 60.0-94.0]. There was also a greater proportion of females in the symptomatic cohort (68.3%, n=398) than in the asymptomatic cohort 55.7% (n=127). Most symptomatic subjects had an ICD-10-CM code of N39.0 for Urinary Tract Infection, site not specified (81.8%, n=534) (Table 1). The asymptomatic cohort specimens were collected from volunteers from the general population and therefore, had no ICD-10-CM codes.

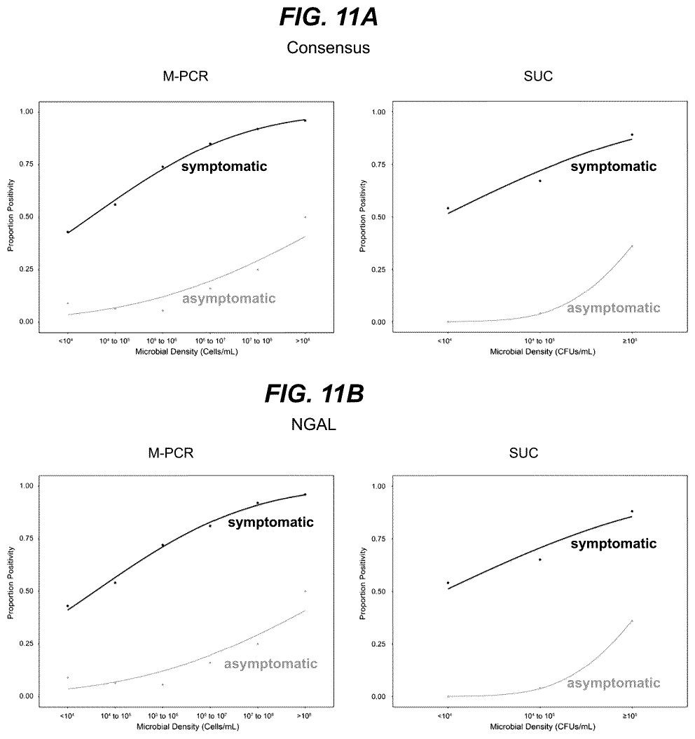

Correlation relationships between biomarker percent positivity and microbial density by M-PCR: First, the correlation between biomarker positivity and microbial density was examined by M-PCR in both urine samples from symptomatic and asymptomatic subjects (FIG. 11A-11D). Each probit regression for symptomatic subjects had an R2>0.90 and a p-value of <0.0001 for all biomarkers in the symptomatic cohort. For the asymptomatic cohort, the probit regressions had R2 values<0.90 for M-PCR microbial densities, but >0.96 for SUC microbial densities and all p-values were <0.05 for all biomarkers, indicating that the correlation between microbial density and biomarker positivity is statistically significant.

Although the symptomatic and asymptomatic cohorts both exhibited a strong positive correlation between biomarker positivity and microbial density, the biomarker proportion positivity was considerably higher across all microbial densities in symptomatic subjects relative to asymptomatic subjects (FIG. 11A-11D).

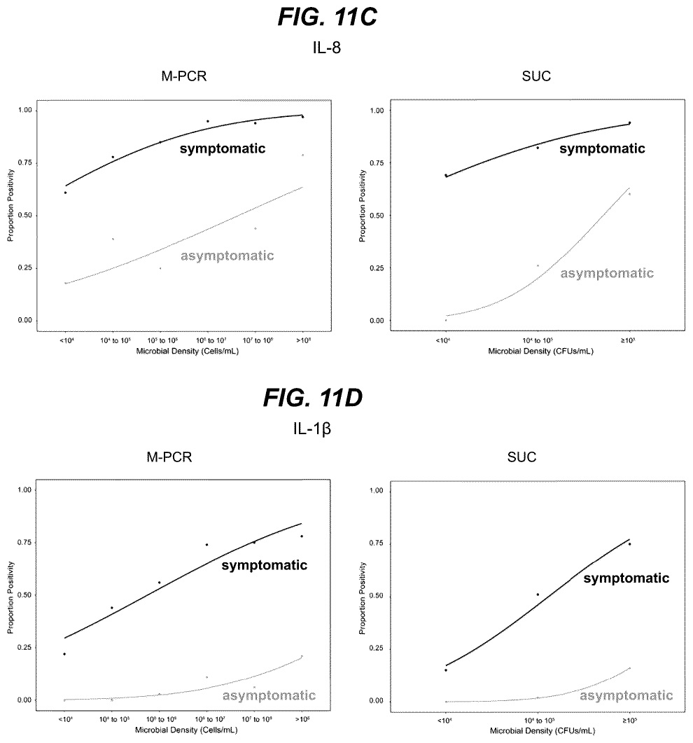

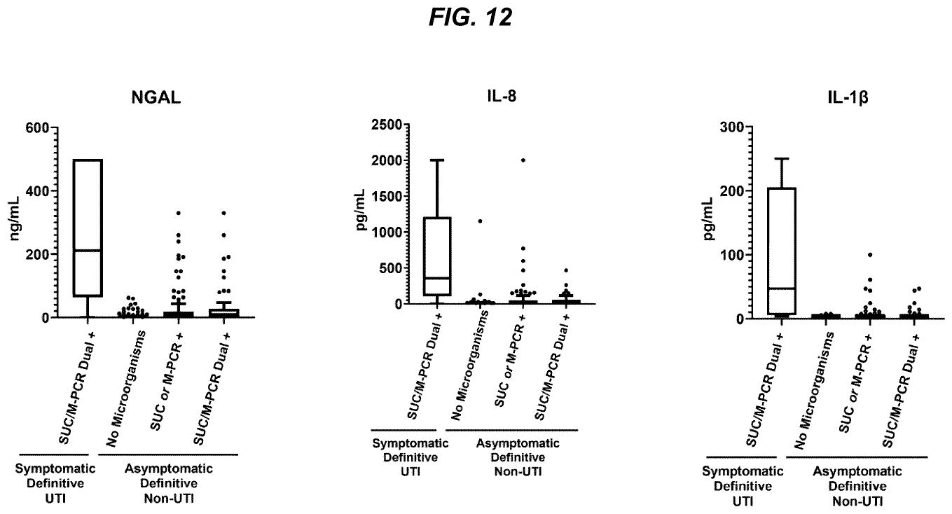

Comparison of biomarker levels between asymptomatic and symptomatic cohorts: Levels of all three biomarkers (NGAL, IL, and IL-1β) are significantly lower (p<0.0001) among all asymptomatic cohort specimens, regardless of the presence of detectable microorganisms (Definitive non-UTIs), compared to the symptomatic cohort specimens with microorganisms detected by both SUC and M-PCR (Definitive UTIs) (FIG. 12 and FIG. 13).

Individual or consensus biomarker positivity in definitive UTIs and definitive non-UTIs: Then, the positivity of the individual biomarkers and combinations of biomarkers were compared against symptomatic Definitive UTI cases and Definitive non-UTIs.

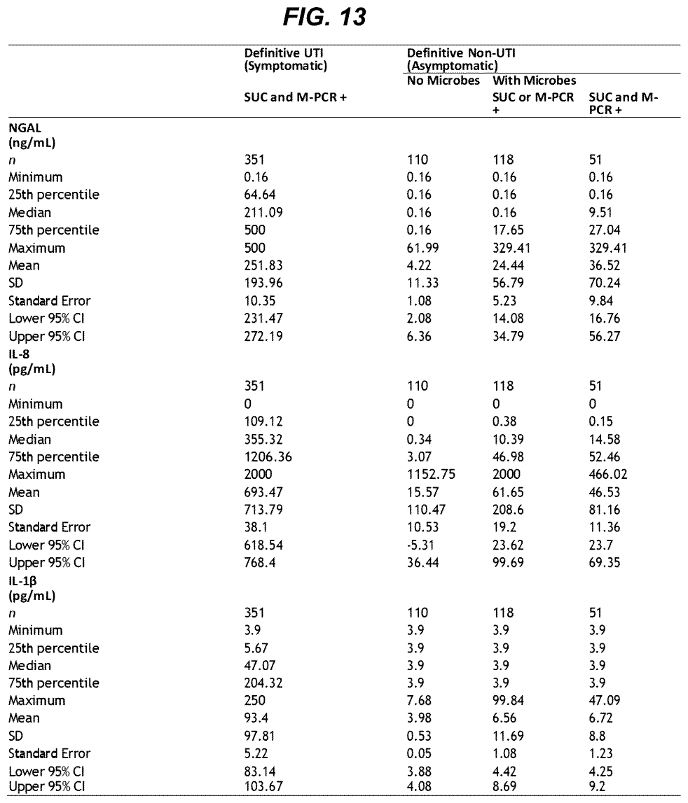

Definitive UTI percentage: Of 583 specimens from symptomatic subjects with a UTI diagnosed in a specialty setting, bacterial detection≥10,000 by both M-PCR (reported in cells/mL) and by SUC (reported in CFUs/mL) occurred in 351 specimens. These 351 specimens were considered Definitive UTI cases. The 228 asymptomatic subject specimens were considered Definitive non-UTI cases regardless of microbial detection, resulting in a 3:2 ratio of Definitive more than half of the asymptomatic group (53.1%, n=122) had detectable microorganisms in the urine at densities>10,000 cells/mL by M-PCR or CFUs/mL by SUC (asymptomatic bacteriuria), and 28.9% had microbial detection at densities>10,000 cells/mL and CFUs/mL by both SUC and M-PCR (n=66) (FIG. 14, Table 18).

TABLE 18
Asymptomatic Subjects with Positive Microbial Detection
(>10,000 CFU or cells/mL for bacteria/bacterial groups
and any cell density for yeasts) by SUC and/or M-PCR.

| | | | SUC or | SUC and - |
|---|---|---|---|---|
| n = 228 | SUC+ | M-PCR+ | M-PCR+ | PCR+ |
| Male (n = 101) | 16 | 21 | 24 | 13 |
| Female (n = 127) | 56 | 94 | 97 | 53 |
| Total (n, %) | 72, 31.6% | 115, 50.4% | 121, 53.1% | 66, 28.9% |

Individual biomarker positivity in distinguishing definitive UTIs and definitive non-UTIs: NGAL was positive in 82.6% (290/351) of definitive UTI cases and negative in 90.8% (207/228) of Definitive non-UTI cases (Table 19). IL-8 was positive in 91.2% (320/351) of Definitive UTI cases and negative in 76.8% (175/228) of definitive non-UTI cases (Table 20). IL-1β was positive in 69.8% (245/351) of definitive UTI cases and negative in 97.9% (221/228) of Definitive non-UTI cases (Table 21). A statistical analysis summary of the three biomarkers is listed in Table 22. IL-8 had the highest sensitivity (91.2%) while IL-1β had the highest specificity (96.9%).

TABLE 19
NGAL Positivity Contingency Table for Criterion 1

| | Definitive UTI | | Definitive non-UTI | Total | |
|---|---|---|---|---|---|
| NGAL Positive | 290 | (50.1%) | 21 (3.6%) | 311 | (53.7%) |
| NGAL Negative | 61 | (10.5%) | 207 (35.8%) | 268 | (46.3%) |
| Total | 351 | (60.6%) | 228 (39.4%) | 579 | (100%) |

TABLE 20
IL-8 Positivity Contingency Table for Criterion 1

| | Definitive UTI | Definitive non-UTI | Total | |
|---|---|---|---|---|
| IL-8 Positive | 320 (55.3%) | 53 (9.1%) | 373 | (64.4%) |
| IL-8 Negative | 31 (5.4%) | 175 (30.2%) | 206 | (35.6%) |
| Total | 351 (60.6%) | 228 (39.4%) | 579 | (100%) |

TABLE 21
IL-1β Positivity Contingency Table for Criterion 1

| | Definitive UTI | Definitive non-UTI | | Total | |
|---|---|---|---|---|---|
| IL-1β Positive | 245 (42.3%) | 7 | (1.2%) | 252 | (43.5%) |
| IL-1β Negative | 106 (18.3%) | 221 | (38.2%) | 327 | (56.5%) |
| Total | 351 (60.6%) | 228 | (39.4%) | 579 | (100%) |

TABLE 22
Biomarker performance comparisons in the
presence of microorganisms based on Criterion 1
Biomarker Performance Characteristics for Differentiating
Definitive UTIs from Definitive non-UTIs

| ≥10,000 Cells/mL | | | |
|---|---|---|---|
| and CFUs/mL | NGAL*** | IL-8*** | IL-1β*** |
| Sensitivity | 82.6% (78.2%, | 91.2% (87.7%, | 69.8% (64.7%, |
| (95% CI) | 86.4%) | 93.9%) | 74.6%) |
| Specificity | 90.8% (86.3%, | 76.8% (70.7%, | 96.9% (93.8%, |
| (95% CI) | 94.2%) | 82.1%) | 98.8%) |
| Positive Predictive | 93.2% (89.9%, | 85.8% (81.8%, | 97.2% (94.4%, |
| Value (95% CI) | 95.8%) | 89.2%) | 98.9%) |
| Negative Predictive | 77.2% (71.7%, | 85.0% (79.3%, | 67.6% (62.2%, |
| Value (95% CI) | 82.1%) | 89.5%) | 72.6%) |
| Accuracy | 85.8% (82.7%, | 85.5% (82.4%, | 80.5% (77.0%, |
| (95% CI) | 88.6%) | 88.3%) | 83.6%) |
| Positive Likelihood | 8.97 (5.95, | 3.92 (3.09, | 22.74 (10.93, |
| Ratio (95% CI) | 13.52) | 4.98) | 47.3) |
| Negative Likelihood | 0.19 (0.13, | 0.12 (0.09, | 0.31 (0.15, |
| Ratio (95% CI) | 0.29) | 0.15) | 0.65) |

***indicates the Proportion Z-test comparison of sensitivity: p-value < 0.0001

“Consensus” or “All three biomarker” positivity in distinguishing definitive UTIs and definitive non-UTIs “Consensus” is defined as two or more biomarkers meeting or exceeding their respective positivity thresholds. “All three biomarkers” is defined as all three biomarkers meeting or exceeding their respective positivity thresholds (Table 11). Consensus positivity occurred in 84.0% (295/351) of Definitive UTI cases and consensus negativity occurred in 91.2% (208/228) of Definitive non-UTI cases (Table 23). All three biomarkers were positive in 66.1% (232/351) of Definitive UTI cases and negative in 97.4% (222/228) of Definitive non-UTI cases (Table 24).

TABLE 23

| | Definitive | Definitive | | |
|---|---|---|---|---|
| | UTI | non-UTI | Total | |
| Consensus Positive | 295 (50.9%) | 20 (3.4%) | 315 | (54.4%) |
| Consensus Negative | 56 (9.7%) | 208 (35.9%) | 264 | (45.6%) |
| Total | 351 (60.6%) | 228 (39.4%) | 579 | (100%) |

TABLE 24
All Three Biomarkers Positivity Contingency Table for Criterion 1

| | Definitive | Definitive | | | |
|---|---|---|---|---|---|
| | UTI | non-UTI | | Total | |
| All Three Positive | 232 (40.1%) | 6 | (1.0%) | 238 | (41.1%) |
| Less than Three Positive | 119 (20.6%) | 222 | (38.3%) | 341 | (58.9%) |
| Total | 351 (60.6%) | 228 | (39.4%) | 579 | (100%) |

A summary of the statistical analysis for the biomarker combinations is listed in Table 25. The consensus criteria of at least two biomarkers meeting or exceeding the positivity threshold performed well in terms of both sensitivity and specificity (84.0% and 91.2%, respectively). Although the combination of all three biomarkers being positive had the highest specificity (97.4%), it had lower sensitivity (66.1%).

TABLE 25
Biomarker “Consensus” and “All three biomarkers”
performance comparisons Based on Criterion 1.
Definitive UTI versus Definitive non-UTI

| ≥10,000 Cells/mL and CFUs/mL | “Consensus”*** | | “All three Biomarkers”*** | |
|---|---|---|---|---|
| Sensitivity (95% CI) | 84.0% | (79.8%, 87.7%) | 66.1% | (60.9%, 71.0%) |
| Specificity (95% CI) | 91.2% | (86.8%, 94.6%) | 97.4% | (94.4%, 99.0%) |
| Positive Predictive Value (95% CI) | 93.7% | (90.4%, 96.1%) | 97.5% | (94.6%, 99.1%) |
| Negative Predictive Value (95% CI) | 78.8% | (73.4%, 83.6%) | 65.1% | (59.8%, 70.2%) |
| Accuracy (95% CI) | 86.9% | (83.8%, 89.5%) | 78.4% | (74.8%, 81.7%) |
| Positive Likelihood Ratio (95% CI) | 9.58 | (6.29, 14.6) | 25.12 | (11.36, 55.51) |
| Negative Likelihood Ratio (95% CI) | 0.17 | (0.11, 0.27) | 0.35 | (0.16, 0.77) |

***indicates the Proportion Z-test comparison of sensitivity: p-value < 0.0001

To determine if the three infection-associated biomarkers selected for this study (NGAL, IL-8, and IL-1β), are both sensitive and specific indicators for UTIs, their levels were measured in both Definitive UTI cases (symptomatic cases, diagnosed in a Urology/Urogynecology specialty setting, with uropathogens identified above threshold values by both SUC and M-PCR) and in Definitive non-UTI control cases (asymptomatic based on FDA-defined criteria included in a Symptom Score Analysis). The Definitive non-UTI cases included asymptomatic individuals with detected microbes (asymptomatic bacteriuria). In this study, more than half of this control group (53.1%, n=121) had had microbial detection at densities≥ 10,000 cells/mL by either SUC or M-PCR, and 28.9% had microbial detection at densities≥10,000 cells/mL by both SUC and M-PCR (n=66) (FIG. 14, Table 18). This relatively high prevalence of microorganisms in urine specimens from the asymptomatic cohort underscores the importance of practicing diagnostic stewardship, such as implementing clinical testing only for the indicated population of symptomatic cases of presumed UTI, and the value of having these types of biomarkers.

In this study of more than 800 subjects, the three biomarkers were significantly elevated in symptomatic subjects with positive microbe identification compared to very low biomarker levels in asymptomatic cases with or without microbe identification. Furthermore, there was a strong positive correlation (R2>0.90; p<0.0001) between microbial density and urine biomarker levels of NGAL, IL-8, and IL-1β for symptomatic subjects. Biomarker “Consensus” (two or more positive biomarkers) exhibited high accuracy in distinguishing definitive UTI from definitive non-UTI cases, with a sensitivity of 90.2%, specificity of 91.2%, positive predictive value (PPV) of 91.7%, negative predictive value (NPV) of 89.7%, and accuracy of 90.7%.

The biomarkers exhibited excellent specificity (>75% individually and >90% for consensus) indicating that urine specimens positive for infection-associated biomarkers are highly likely to be associated with cases of active UTIs. There was also a strong correlation between microbe density and rising positivity levels, with high positivity levels in symptomatic patients appearing even at 10,000 cells/mL and CFU/mL in symptomatic patients. Positivity levels for asymptomatic cases remained low even at 100,000 cells/mL and CFU/mL, though there was some increase observed with rising microbe density.

The high sensitivity and specificity (>90%) of the “Consensus” biomarker model for UTIs make it a valuable tool to differentiate true UTI cases from asymptomatic bacteriuria and other false-positive differential diagnoses, and also for establishing an objective “truth” for the comparison of existing and novel diagnostic test accuracy. This is especially important since the current “gold standard” test, SUC, is known to have significant limitations, making it an unreliable source of diagnostic “truth.”

The measurement of urinary biomarkers, individually or in combination, may also prove valuable as a supportive tool for the clinical diagnostic workup of suspected UTIs, especially in patients unable to clearly communicate their symptoms, such as pediatric patients and patients with cognitive impairment. Leukocyte esterase (LE) dipstick analysis is often employed in clinics as part of the diagnostic workup for UTI, even though the specificity is usually too low to be useful as an individual test (sensitivity range 72-94%; specificity range 9-59%). The contrasting high accuracy of the consensus biomarker model detailed here indicates it could be a superior tool for assisting in the diagnosis of UTI.

Using symptomatic subjects' urine specimens in which SUC and M-PCR results agreed on the presence of uropathogens, the association of NGAL, IL-8, and IL-1β, with Definitive UTI cases was demonstrated. A consensus criterion with >2 of the biomarkers meeting the positivity thresholds showed a good balance of sensitivity (84.0%), specificity (91.2%), and accuracy (86.9%), making it an excellent supportive diagnostic tool for resolving the presence of active UTI, particularly if SUC and M-PCR results disagree. These biomarkers can be used as an important supplemental tool to determine if a case is a UTI when the microbial detection and identification diagnostic test has significant limitations in sensitivity or when it is unclear whether the detected microorganism(s) is causing disease.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from a 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®.NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

### EMBODIMENTS

The following embodiments are intended to be illustrative only and not to be limiting in any way.

Embodiment 1: An in vitro method comprising: a) obtaining or having obtained a urine sample from a subject having or suspected of having a urinary tract infection; and b) detecting levels of at least two biomarker proteins selected from a group consisting of neutrophil gelatinase-associated lipocalin (NGAL), interleukin-1β (IL-1β), and IL-8; wherein if the level of the at least two biomarker proteins are higher than a reference level, then the subject has a urinary tract infection. Embodiment 2: The method of embodiment 1, wherein the in vitro method distinguishes asymptomatic bacteriuria and a urinary tract infection (UTI) in the patient. Embodiment 3: The method of embodiment 1 or embodiment 2, wherein the subject is experiencing atypical symptoms.

Embodiment 4: An in vitro method for distinguishing between an asymptomatic bacteriuria and a urinary tract infection (UTI) in a subject having or suspected of having a urinary tract infection, the method comprising: a) obtaining or having obtained a urine sample from the subject; and b) detecting levels at least two biomarker proteins selected from a group consisting of neutrophil gelatinase-associated lipocalin (NGAL), interleukin-1β (IL-1β), and IL-8; wherein if the level of the at least two biomarker proteins are higher than a reference level, then the subject has a UTI. Embodiment 5: An in vitro method for the diagnosis of a urinary tract infection (UTI) in a subject, comprising the steps of: a) providing a urine sample from the subject; and b) detecting the levels of at least two biomarker proteins selected from a group consisting of neutrophil gelatinase-associated lipocalin (NGAL), interleukin-1β (IL-1β), and IL-8 in the urine sample; wherein a higher level of at least two of the biomarkers in the urine sample as determined in step b) compared to a reference level is indicative of a UTI in the subject.

Embodiment 6: An in vitro method for the diagnosis of a urinary tract infection (UTI) in a subject, comprising the steps of: a) providing a urine sample from the subject; and b) detecting the levels of three biomarker proteins selected from a group consisting of neutrophil gelatinase-associated lipocalin (NGAL), interleukin-1β (IL-1β), and IL-8 in the urine sample; wherein a higher level of at least two of the biomarkers in the urine sample as determined in step b) compared to a reference level is indicative of a UTI in the subject.

Embodiment 7: The method of any one of embodiments 1-6, wherein the patient is an adult subject. Embodiment 8: The method of any one of embodiments 1-6, wherein the subject is a pediatric subject. Embodiment 9: The method of any one of embodiments 1-6, wherein the subject is a geriatric subject.

Embodiment 10: The method of any one of embodiments 1-9, wherein the urine sample is a voided urine sample. Embodiment 11: The method of any one of embodiments 1-9, wherein the urine sample is a catheterized urine sample. Embodiment 12: The method of any one of embodiments 1-9, wherein the urine sample is obtained from an undergarment or a diaper.

Embodiment 13: The method of any one of embodiments 1-12, wherein the reference level of NGAL is about >38.0 ng/ml. Embodiment 14: The method of any one of embodiments 1-12, wherein the reference level of IL-8 is about >20.6 pg/mL. Embodiment 15: The method of any one of embodiments 1-12, wherein the reference level of IL-1β is about ≥12.4 pg/mL.

Embodiment 16: The method of any one of embodiments 1-15, wherein the biomarkers are detected by a Western blot, dot blot, an ELISA, a lateral flow assay, or mass spectrometry. Embodiment 17: The method of any one of embodiments 1-16, wherein the biomarkers are detected using one or more antibodies. Embodiment 18: The method of any one of embodiments 1-17, wherein the biomarkers are detected using microfluidics. Embodiment 19: The method of any one of embodiments 1-18 further comprising detecting one or a combination of nitrites, carbohydrates, white blood cells (WBCs), or red blood cells (RBCs).

Embodiment 20: A detection system comprising a) a sample receiving zone to receive a sample from a subject having or suspected of having a urinary tract infection and b) at least two detection zones for detecting at least two biomarker proteins selected from a group consisting of neutrophil gelatinase-associated lipocalin (NGAL), interleukin-1β (IL-1β), and IL-8. Embodiment 21: A detection system comprising a) a sample receiving zone to receive a sample from a subject having or suspected of having a urinary tract infection and b) three detection zones for detecting biomarker proteins selected from a group consisting of neutrophil gelatinase-associated lipocalin (NGAL), interleukin-1β (IL-1β), and IL-8.

Embodiment 22: The detection system of embodiment 20 or embodiment 21, wherein the subject is experiencing atypical symptoms. Embodiment 23: The detection system of embodiment 20 or embodiment 21, wherein the patient is an adult patient. Embodiment 24: The detection system of embodiment 20 or embodiment 21, wherein the subject is a pediatric subject. Embodiment 25: The detection system of embodiment 20 or embodiment 21, wherein the subject is a geriatric subject.

Embodiment 26: The detection system of any one of embodiments 20-25, wherein the sample is a urine sample. Embodiment 27: The detection system of any one of embodiments 20-26, wherein the urine sample is a voided urine sample. Embodiment 28: The detection system of any one of embodiments 20-26, wherein the urine sample is a catheterized urine sample. Embodiment 29: The detection system of any one of embodiments 20-26, wherein the urine sample is obtained from an undergarment or a diaper.

Embodiment 30: The detection system of any one of embodiments 20-29, wherein each of the detection zones comprise antibodies for binding the biomarker proteins. Embodiment 31: The detection system of any one of embodiments 20-30, wherein the system is a biochip, a test strip, a microtiter plate, or a microfluidic plate. Embodiment 32: The detection system of any one of embodiments 20-31, wherein the biomarker proteins are detected using a lateral flow assay or an ELISA. Embodiment 33: The method of any one of embodiments 20-32 further comprising detecting one or a combination of nitrites, carbohydrates, white blood cells (WBCs), or red blood cells (RBCs). Embodiment 34: A kit comprising a detection system according to any one of embodiments 20-33.

Embodiment 35: A detection system for detecting a urinary tract infection (UTI) in a subject having or suspected of having a UTI, said system configured to detect the presence of at least two of neutrophil gelatinase-associated lipocalin (NGAL), interleukin-1β (IL-1β), and IL-8 in a urine sample from the subject, wherein detection of at least two of NGAL, IL-1β, and IL-8 above a reference level is indicative of a UTI. Embodiment 36: A detection system for detecting a urinary tract infection (UTI) in a subject having or suspected of having a UTI, said system configured to detect the presence of neutrophil gelatinase-associated lipocalin (NGAL), interleukin-1β (IL-1β), and IL-8 in a urine sample from the subject, wherein detection of at least two of NGAL, IL-1β, and IL-8 above a reference level is indicative of a UTI.

Embodiment 37: The detection system of embodiment 35 or embodiment 36, wherein the subject is experiencing atypical symptoms. Embodiment 38: The detection system of any one of embodiments 35-37, wherein the patient is an adult patient. Embodiment 39: The detection system of any one of embodiments 35-37, wherein the subject is a pediatric subject. Embodiment 40: The detection system of any one of embodiments 35-37, wherein the subject is a geriatric subject.

Embodiment 41: The detection system of any one of embodiments 35-40, wherein the urine sample is a voided urine sample. Embodiment 42: The detection system of any one of embodiments 35-41, wherein the urine sample is a catheterized urine sample. Embodiment 43: The detection system of any one of embodiments 35-41, wherein the urine sample is obtained from an undergarment or a diaper.

Embodiment 44: The detection system of any one of embodiments 35-43, wherein the system is a biochip, a test strip, a microtiter plate, or a microfluidic plate. Embodiment 45: The detection system of any one of embodiments 35-44, wherein the biomarker proteins are detected using a lateral flow assay or an ELISA. Embodiment 46: The detection system of any one of embodiments 35-45, wherein the system is further configured to detect one or a combination of nitrites, carbohydrates, white blood cells (WBCs), or red blood cells (RBCs). Embodiment 47: A kit comprising a detection system according to any one of embodiments 35-46.

Embodiment 48: A system for detecting a urinary tract infection (UTI) in a subject, the system comprising: a) a sample receiving zone to which a urine sample from the subject is added; b) a conjugating zone positioned downstream from the sample receiving zone, said conjugating zone comprising at least two mobilizable signal producing component, wherein each mobilizable signal producing component binds to one of the biomarkers selected from a group consisting of neutrophil gelatinase-associated lipocalin (NGAL), IL-1β, and IL-8; c) at least two detection zones positioned downstream from the conjugating zone, wherein each detection zone comprises a component that restrains each biomarker conjugated to a mobilizable signal producing component, and d) a control zone positioned downstream from the detection zone, the control zone comprising an immobilized component that binds with the labeled control reagent.

Embodiment 49: A system for detecting a urinary tract infection (UTI) in a subject, the system comprising: a) a sample receiving zone to which a urine sample from the subject is added; b) a conjugating zone positioned downstream from the sample receiving zone, said conjugating zone comprising at least three mobilizable signal producing component, wherein each mobilizable signal producing component binds to one of the biomarkers selected from a group consisting of neutrophil gelatinase-associated lipocalin (NGAL), interleukin-1 (IL-1β), and IL-8; c) at least three detection zones positioned downstream from the conjugating zone, wherein each detection zone comprises a component that restrains each biomarker conjugated to a mobilizable signal producing component, and d) a control zone positioned downstream from the detection zone, the control zone comprising an immobilized component that binds with the labeled control reagent.

Embodiment 50: The system of embodiment 48 or embodiment 49, wherein the mobilizable signal producing component is selected from the group consisting of a chemiluminescent agent, a particulate label, a colorimetric agent, an energy transfer agent, an enzyme, a fluorescent agent and a radioisotope. Embodiment 51: The system of embodiment 48 or embodiment 49, wherein the mobilizable signal producing component is a fluorescent agent; wherein the fluorescent agent is a fluorescent probe.

Embodiment 52: The system of any one of embodiments 48-51, wherein each detection zone comprises an antibody that binds the biomarker. Embodiment 53: The system of any one of embodiments 48-52, wherein the immobilized component in the control zone comprises an antibody. Embodiment 54: The system of embodiment 53, wherein the antibody in the control zone is specific for a labeled control reagent.

Embodiment 55: The system of any one of embodiments 48-54, wherein the system is a biochip, a test strip, a microtiter plate, or a microfluidic plate.

Embodiment 56: The system of any one of embodiments 48-55, wherein the subject is experiencing atypical symptoms. Embodiment 57: The system of any one of embodiments 48-56, wherein the patient is an adult patient. Embodiment 58: The system of any one of embodiments 48-56, wherein the subject is a pediatric subject. Embodiment 59: The system of any one of embodiments 48-56, wherein the subject is a geriatric subject.

Embodiment 60: The system of any one of embodiments 48-59, wherein the urine sample is a voided urine sample. Embodiment 61: The system of any one of embodiments 48-59, wherein the urine sample is a catheterized urine sample. Embodiment 62: The system of any one of embodiments 48-59, wherein the urine sample is obtained from an undergarment or a diaper.

Embodiment 63: The system of any one of embodiments 48-62, wherein the system is configured to detect one or a combination of nitrites, carbohydrates, white blood cells (WBCs), or red blood cells (RBCs). Embodiment 64: The system of any one of embodiments 48-63, wherein the system is configured to present a result of either UTI or asymptomatic bacteriuria. Embodiment 65: The system of embodiment 64, wherein the result is displayed as a composite result.

Embodiment 66: A lateral flow system for detecting a urinary tract infection (UTI) in a patient, said system comprising a lateral flow assay for a urine sample, wherein the lateral flow assay is configured to detect the presence of at least two of neutrophil gelatinase-associated lipocalin (NGAL), interleukin-1β (IL-1β), and IL-8 in the urine sample, wherein detection of at least two of NGAL, IL-1β, and IL-8 above a reference level is indicative of a UTI.

Embodiment 67: A lateral flow system for detecting a urinary tract infection (UTI) in a patient, said system comprising a lateral flow assay for a urine sample, wherein the lateral flow assay is configured to detect the presence of neutrophil gelatinase-associated lipocalin (NGAL), interleukin-1β (IL-1β), and IL-8 in the urine sample, wherein detection of at least two of NGAL, IL-1β, and IL-8 above a reference level is indicative of a UTI.

Embodiment 68: The system of embodiment 66 or embodiment 67, wherein the system is able to distinguish asymptomatic bacteriuria and a urinary tract infection (UTI) in the patient.

Embodiment 69: A lateral flow system for distinguishing between asymptomatic bacteriuria and a urinary tract infection (UTI) in a patient, said system comprising: a lateral flow assay for a urine sample, wherein the lateral flow assay is configured to detect the presence of at least two of neutrophil gelatinase-associated lipocalin (NGAL) and interleukin-1β (IL-1β), and IL-8 in the urine sample, wherein detection of at least two of neutrophil gelatinase-associated lipocalin (NGAL), IL-1β, and IL-8 is indicative of a UTI.

Embodiment 70: A lateral flow system for distinguishing between asymptomatic bacteriuria and a urinary tract infection (UTI) in a patient, said system comprising: a lateral flow assay for a urine sample, wherein the lateral flow assay is configured to detect the presence of neutrophil gelatinase-associated lipocalin (NGAL), interleukin-1β (IL-1β), and IL-8 in the urine sample, wherein detection of at least two of neutrophil gelatinase-associated lipocalin (NGAL), IL-1β, and IL-8 is indicative of a UTI.

Embodiment 71: The lateral flow system of any one of embodiments 66-70, wherein the subject is experiencing atypical symptoms. Embodiment 72: The lateral flow system of any one of embodiments 66-71, wherein the patient is an adult patient. Embodiment 73: The lateral flow system of any one of embodiments 66-71, wherein the subject is a pediatric subject. Embodiment 74: The lateral flow system of any one of embodiments 66-71, wherein the subject is a geriatric subject.

Embodiment 75: The lateral flow system of any one of embodiments 66-74, wherein the urine sample is a voided urine sample. Embodiment 76: The lateral flow system of any one of embodiments 66-74, wherein urine sample is a catheterized urine sample. Embodiment 77: The lateral flow system of any one of embodiments 66-74, wherein the urine sample is obtained from an undergarment or a diaper.

Embodiment 78: The lateral flow system of any one of embodiments 66-77 wherein the system is a biochip, a test strip, a microtiter plate, or a microfluidic plate. Embodiment 79: The lateral flow system of any one of embodiments 66-78, wherein the biomarker proteins are detected using a lateral flow assay or an ELISA. Embodiment 80: lateral flow system of any one of embodiments 66-79, wherein the system is further configured to detect one or a combination of nitrites, carbohydrates, white blood cells (WBCs), or red blood cells (RBCs). Embodiment 81: A kit comprising a lateral flow system according to any one of embodiments 66-80.

Embodiment 82: A method of distinguishing between a urinary tract infection (UTI) and asymptomatic bacteriuria in a patient, said method comprising: introducing a portion of a urine sample derived from the patient to a lateral flow assay, the lateral flow assay is configured to detect the presence of at least two of neutrophil gelatinase-associated lipocalin (NGAL) interleukin-1β (IL-1β) and IL-8 in the urine sample, wherein detection of at least two of neutrophil gelatinase-associated lipocalin (NGAL), IL-1β, and IL-8 is indicative of a UTI.

Embodiment 83: A method of distinguishing between a urinary tract infection (UTI) and asymptomatic bacteriuria in a patient, said method comprising: introducing a portion of a urine sample derived from the patient to a lateral flow assay, the lateral flow assay is configured to detect the presence of neutrophil gelatinase-associated lipocalin (NGAL) interleukin-1β (IL-1β), and IL-8 in the urine sample, wherein detection of at least two of neutrophil gelatinase-associated lipocalin (NGAL), IL-1β, and IL-8 is indicative of a UTI.

Embodiment 84: The method of embodiment 82 or embodiment 83, wherein the subject is experiencing atypical symptoms. Embodiment 85: The method of any one of embodiments 82-84, wherein the patient is an adult patient. Embodiment 86: The method of any one of embodiments 82-84, wherein the subject is a pediatric subject. Embodiment 87: The method of any one of embodiments 82-84, wherein the subject is a geriatric subject.

Embodiment 88: The method of any one of embodiments 82-87, wherein the urine sample is a voided urine sample. Embodiment 89: The method of any one of embodiments 82-87, wherein the urine sample is a catheterized urine sample. Embodiment 90: The method of any one of embodiments 82-87, wherein the urine sample is obtained from an undergarment or a diaper.

Embodiment 91: The method of any one of embodiments 82-90, wherein the system is a biochip, a test strip, a microtiter plate, or a microfluidic plate. Embodiment 92: The method of any one of embodiments 82-91, wherein the biomarker proteins are detected using a lateral flow assay or an ELISA. Embodiment 93: The method of any one of embodiments 82-92, wherein the system is further configured to detect one or a combination of nitrites, carbohydrates, white blood cells (WBCs), or red blood cells (RBCs).

Embodiment 94: A method of measuring the levels of biomarker proteins in a biological sample from a subject having or suspected of having a urinary tract infection (UTI), the method comprising, a) measuring levels of of at least two biomarker proteins selected from a group consisting of neutrophil gelatinase-associated lipocalin (NGAL), interleukin-1β (IL-1β), and IL-8; b) detecting a changed level of NGAL, IL-1β, and IL-8 relative to a reference level of NGAL, IL-1β, and IL-8; and b) administering or having administered a treatment for a UTI when an increased level of at least two biomarkers are detected.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A detection system for detecting a urinary tract infection (UTI) in a subject having or suspected of having a urinary tract infection, the system comprising:
a) a sample receiving zone to receive a portion of a urine sample from the subject; and
b) at least two detection zones for detecting at least two biomarker proteins selected from a group consisting of neutrophil gelatinase-associated lipocalin (NGAL), interleukin-1β (IL-1β), and IL-8,
wherein detection of at least two biomarker proteins above a reference level is indicative of a UTI.

2. The detection system of claim 1, wherein the subject is experiencing atypical symptoms.

3. The detection system of claim 1, wherein the subject is a pediatric subject or a geriatric subject.

4. The detection system of claim 1, wherein each of the detection zones comprises antibodies for binding the biomarker proteins.

5. The detection system of claim 1, wherein the system is a biochip, a test strip, a microtiter plate, or a microfluidic plate.

6. The detection system of claim 1, wherein the biomarker proteins are detected using a lateral flow assay or an ELISA.

7. The detection system of claim 1, wherein the reference level of NGAL is about 38 ng/ml, the reference level of IL-8 is about 20 pg/mL, and the reference level of IL-1β is about 12 pg/mL.

8. The detection system of claim 1 further comprises one or more sensors operatively coupled to the detection zones, wherein the one or more sensors are configured to detect a change in the detection zones in response to the sample, generate a signal based on the change in the detection zones, and transmit the signal to a computing device.

9. The detection system of claim 7, wherein the change in the detection zones is a change in color, fluorescence, presence of a protein, electrical current, or a combination thereof.

10. The detection system of claim 1 further comprising a control zone configured to bind a control protein.

11. A method of detecting a urinary tract infection (UTI) in a subject having or suspected of having a urinary tract infection, said method comprising: introducing a portion of a urine sample derived from the patient to a detection system comprising:
a) a sample receiving zone to receive the portion of the urine sample from the subject; and
b) at least two detection zones for detecting at least two biomarker proteins selected from a group consisting of neutrophil gelatinase-associated lipocalin (NGAL), interleukin-1β (IL-1β), and IL-8;
wherein detection of at least two biomarker proteins above a reference level is indicative of a UTI.

12. The method of claim 11, wherein the subject is experiencing atypical symptoms.

13. The method of claim 11, wherein the subject is a pediatric subject or a geriatric subject.

14. The method of claim 11, wherein the detection system is a biochip, a test strip, a microtiter plate, or a microfluidic plate.

15. The method of claim 11, wherein the biomarker proteins are detected using a lateral flow assay or an ELISA.

16. The method of claim 11, wherein the reference level of NGAL is about 38 ng/ml, the reference level of IL-8 is about 20 pg/mL, and the reference level of IL-1β is about 12 pg/mL.

17. The method of claim 11 further comprises one or more sensors operatively coupled to the detection zones, wherein the one or more sensors are configured to detect a change in the detection zones in response to the sample, generate a signal based on the change in the detection zones, and transmit the signal to a computing device.

18. The method of claim 17, wherein the change in the detection zones is a change in color, fluorescence, presence of a protein, electrical current, or a combination thereof.

19. The method of claim 11 further comprising a control zone configured to bind a control protein.

20. The method of claim 11, wherein the method distinguishes asymptomatic bacteriuria and a urinary tract infection (UTI) in the subject.
